SEQUENCE LISTING (4 sequences)

SEQ ID NO: 1 - DNA, 2579 bp, Homo sapiens
gaattcaccaccatgcttggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcacgcgtttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggttagttatggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagcttctagagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtctcgaggtgctcgcggcagtgccaggagggccaggtgcgccgggtcaaggggttccactcctgctgctacgactgtgtggactgcgaggcgggcagctaccggcaaaatccggacgatatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtgagcggccgc

SEQ ID NO: 2 - protein, 856 aa, Homo sapiens
EFTTMLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 3 - DNA, 2577 bp, Mus musculus
atgccagctttggctatcatgggtctcagcctggctgctttcctggagcttgggatgggggcctctttgtgtctgtcacagcaattcaaggcacaaggggactacatactgggcgggctatttcccctgggctcaaccgaggaggccactctcaaccagagaacacaacccaacagcatcccgtgcaacaggttctcaccccttggtttgttcctggccatggctatgaagatggctgtggaggagatcaacaatggatctgccttgctccctgggctgcggctgggctatgacctatttgacacatgctccgagccagtggtcaccatgaaatccagtctcatgttcctggccaaggtgggcagtcaaagcattgctgcctactgcaactacacacagtaccaaccccgtgtgctggctgtcatcggcccccactcatcagagcttgccctcattacaggcaagttcttcagcttcttcctcatgccacaggtcagctatagtgccagcatggatcggctaagtgaccgggaaacgtttccatccttcttccgcacagtgcccagtgaccgggtgcagctgcaggcagttgtgactctgttgcagaacttcagctggaactgggtggccgccttagggagtgatgatgactatggccgggaaggtctgagcatcttttctagtctggccaatgcacgaggtatctgcatcgcacatgagggcctggtgccacaacatgacactagtggccaacagttgggcaaggtgctggatgtactacgccaagtgaaccaaagtaaagtacaagtggtggtgctgtttgcctctgcccgtgctgtctactccctttttagttacagcatccatcatggcctctcacccaaggtatgggtggccagtgagtcttggctgacatctgacctggtcatgacacttcccaatattgcccgtgtgggcactgtgcttgggtttttgcagcggggtgccctactgcctgaattttcccattatgtggagactcaccttgccctggccgctgacccagcattctgtgcctcactgaatgcggagttggatctggaggaacatgtgatggggcaacgctgtccacggtgtgacgacatcatgctgcagaacctatcatctgggctgttgcagaacctatcagctgggcaattgcaccaccaaatatttgcaacctatgcagctgtgtacagtgtggctcaagcccttcacaacaccctacagtgcaatgtctcacattgccacgtatcagaacatgttctaccctggcagctcctggagaacatgtacaatatgagtttccatgctcgagacttgacactacagtttgatgctgaagggaatgtagacatggaatatgacctgaagatgtgggtgtggcagagccctacacctgtattacatactgtgggcaccttcaacggcacccttcagctgcagcagtctaaaatgtactggccaggcaaccaggtgccagtctcccagtgttcccgccagtgcaaagatggccaggttcgccgagtaaagggctttcattcctgctgctatgactgcgtggactgcaaggcgggcagctaccggaagcatccagatgacttcacctgtactccatgtaaccaggaccagtggtccccagagaaaagcacagcctgcttacctcgcaggcccaagtttctggcttggggggagccagttgtgctgtcactcctcctgctgctttgcctggtgctgggtctagcactggctgctctggggctctctgtccaccactgggacagccctcttgtccaggcctcaggtggctcacagttctgctttggcctgatctgcctaggcctcttctgcctcagtgtccttctgttcccagggcggccaagctctgccagctgccttgcacaacaaccaatggctcacctccctctcacaggctgcctgagcacactcttcctgcaagcagctgagacctttgtggagtctgagctgccactgagctgggcaaactggctatgcagctaccttcggggactctgggcctggctagtggtactgttggccacttttgtggaggcagcactatgtgcctggtatttgatcgctttcccaccagaggtggtgacagactggtcagtgctgcccacagaggtactggagcactgccacgtgcgttcctgggtcagcctgggcttggtgcacatcaccaatgcaatgttagctttcctctgctttctgggcactttcctggtacagagccagcctggccgctacaaccgtgcccgtggtctcaccttcgccatgctagcttatttcatcacctgggtctcttttgtgcccctcctggccaatgtgcaggtggcctaccagccagctgtgcagatgggtgctatcctagtctgtgccctgggcatcctggtcaccttccacctgcccaagtgctatgtgcttctttggctgccaaagctcaacacccaggagttcttcctgggaaggaatgccaagaaagcagcagatgagaacagtggcggtggtgaggcagctcagggacacaatgaatga

SEQ ID NO: 4 - protein, 858 aa, Mus musculus
MPALAIMGLSLAAFLELGMGASLCLSQQFKAQGDYILGGLFPLGSTEEATLNQRTQPNSIPCNRFSPLGLFLAMAMKMAVEEINNGSALLPGLRLGYDLFDTCSEPVVTMKSSLMFLAKVGSQSIAAYCNYTQYQPRVLAVIGPHSSELALITGKFFSFFLMPQVSYSASMDRLSDRETFPSFFRTVPSDRVQLQAVVTLLQNFSWNWVAALGSDDDYGREGLSIFSSLANARGICIAHEGLVPQHDTSGQQLGKVLDVLRQVNQSKVQVVVLFASARAVYSLFSYSIHHGLSPKVWVASESWLTSDLVMTLPNIARVGTVLGFLQRGALLPEFSHYVETHLALAADPAFCASLNAELDLEEHVMGQRCPRCDDIMLQNLSSGLLQNLSAGQLHHQIFATYAAVYSVAQALHNTLQCNVSHCHVSEHVLPWQLLENMYNMSFHARDLTLQFDAEGNVDMEYDLKMWVWQSPTPVLHTVGTFNGTLQLQQSKMYWPGNQVPVSQCSRQCKDGQVRRVKGFHSCCYDCVDCKAGSYRKHPDDFTCTPCNQDQWSPEKSTACLPRRPKFLAWGEPVVLSLLLLLCLVLGLALAALGLSVHHWDSPLVQASGGSQFCFGLICLGLFCLSVLLFPGRPSSASCLAQQPMAHLPLTGCLSTLFLQAAETFVESELPLSWANWLCSYLRGLWAWLVVLLATFVEAALCAWYLIAFPPEVVTDWSVLPTEVLEHCHVRSWVSLGLVHITNAMLAFLCFLGTFLVQSQPGRYNRARGLTFAMLAYFITWVSFVPLLANVQVAYQPAVQMGAILVCALGILVTFHLPKCYVLLWLPKLNTQEFFLGRNAKKAADENSGGGEAAQGHNE

(19) United States
(12) Patent Application Publication
(10) Pub. No.: US 2012/0245213 A1
(43) Pub. Date: Sep. 27, 2012

(54) HUMAN TYPE I TASTE RECEPTOR SUBUNIT 3 MODULATORS AND METHODS OF USING SAME

(76) Inventors: Bedrich Mosinger, Cherry Hill, NJ (US); Emeline Maillet, Bagneux (FR); Robert Margolskee, Voorhees, NJ (US)

(21) Appl. No.: 13/499,885

(22) PCT Filed: Oct. 1, 2010
(86) PCT No.: PCT/US10/51135

§ 371 Date: Jun. 12, 2012

Related U.S. Application Data
(60) Provisional application No. 61/247,823, filed on Oct. 1, 2009.

(51) Int. Cl. A61K 31/405; A61K 31/192; C07C 57/30; C07C 57/40; C07C 59/68; A61P 1/18; C07C 229/34; C07D 209/18; A61P 3/06; A61P 3/10; A61P 9/12; A61P 3/00; G01N 21/64; A61K 31/195

(52) U.S. Cl. 514/419; 435/7.21; 514/570; 562/496; 514/569; 562/490; 562/472; 514/571; 514/563; 562/451; 548/494

(57) ABSTRACT

Methods and compositions are provided for modulating the activity of human type I taste receptor subunit 3 (hT1R3). Such materials and methods are useful for the screening and preparation of compositions and methods for the treatment of carbohydrate and lipid metabolic diseases and disorders.

This application claims priority to U.S. Provisional Application No. 61/247,823 filed Oct. 1, 2009.

This invention was made with U.S. government support under grant numbers 1 R21 DC007399-01A1, 5 R03 DC007984-02, DK073248, DC008301, and DC007984 awarded by the National Institutes of Health. The government has certain rights in the invention.

### FIELD OF THE INVENTION

The present disclosure relates to materials and methods for modulating the activity of human type I taste receptor subunit 3 (hT1R3).

### BACKGROUND OF THE INVENTION

The type I taste receptors (T1R5) are G-protein-coupled receptors that underlie sweet and umami (amino acid) taste (Brauner-Osborne, H., et al; Curr Drug Targets 2007, 8, 169-84). The T1R2+T1R3 heterodimer responds to sugars and sweeteners; T1R1+T1R3 responds to glutamate and other amino acids (Zhao, G. Q. et al., Cell 2003, 115, 255-66; Max, M. and Meyerhof, W., The Senses: A Comprehensive Reference, Allan I Basbaum, A. K., Gordon M Shepherd and Gerald Westheimer, Ed. Stuart Firestein and Gary K. Beauchamp. San Diego: Academic Press: 2008; Vol. 4, Olfaction & Taste, pp 197-218). T1R3 alone, possibly as a homodimer, may serve as a low-affinity sweet receptor for carbohydrates (Max, supra). T1R receptors, the taste G protein gustducin and other taste transduction proteins are expressed in taste cells of the tongue and in a number of non-taste tissues including enteroendocrine cells of the gastrointestinal tract and pancreatic islets (Stermini, C., et al., Curr Opin Endocrinol Diabetes Obes 2008, 15, 73-78; Toyono, T., et al., Biochim Biophys Acta 2007, 1769, 641-648).

Sugars and artificial sweeteners are powerful agonists of the sweet taste receptors of both tongue and gut (Xu, H., et al., Proc Natl Acad Sci USA 2004, 101, 14258-14263; Jang, H. J., et al., Proc Natl Acad Sci USA 2007, 104, 15069-15074; and Mace, O. J., et al., J Physiol 2007, 582, 379-392). Activated sweet receptors in taste cells signal the presence of carbohydrate-rich foods to the brain; the same receptors in intestinal enteroendocrine cells regulate secretion of glucagon-like peptide-1 (GLP-1) and induce expression of sodium-glucose co-transporter-1 (SGLT1) leading to enhanced absorption of carbohydrates (Egan, J. M., and Margolskee, R. F., Mol Interv 2008, 8, 78-81; Margolskee, R. F., et al., Proc Natl Acad Sci USA 2007, 104, 15075-15080). Knockout mice lacking gustducin are deficient in detecting sweet and umami compounds and have dysregulated glucose homeostasis (Jang, supra; and Egan, supra). Yet little attention has been paid to the physiological effects of artificial sweeteners beyond their sweet taste. Studies now indicate that artificial sweeteners activate intestinal T1R receptors (Jang, supra, Mace, supra, Margolskee, supra). Of potential relevance is the observation that ingestion of diet soda is associated with an increased risk of metabolic syndrome, thereby increasing the risk for heart disease, stroke, and diabetes (Lutsey, P. L., et al., Circulation 2008, 117, 754-761). These studies indicate that taste receptors and other taste signaling proteins expressed in gut and other endocrine organs may have an important role in glucose homeostasis and energy metabolism and that their altered activity may contribute to pathologies such as type II diabetes and obesity.

A number of naturally occurring anti-sweet or sweet-modifying substances are suspected to be ligands of the sweet receptor, but to date the site(s) of action of only a few of these compounds have been identified (Kanetkar, P., et al., J Clin Biochem Nutr 2007, 41, 77-81; Kurihara, Y., Crit. Rev Food Sci Nutr 1992, 32, 231-252; and Jiang, P., et al., J Biol Chem 2005, 280, 15238-15246). The best-described inhibitor of sweet taste is lactisole (methoxy-phenoxy-propionic acid), originally isolated from coffee beans (Flament, I., In Coffee flavor chemistry, ed., J. W. a. S., Ed. p p 207). Lactisole's site of action has been mapped to the transmembrane domain of human T1R3 (Xu, supra, Jiang, supra, and Winnig, M., et al., BMC Neurosci 2005, 6, 22). This region of T1R3 receptors is well conserved in humans, old world monkeys and primates, but differs in other species. Indeed, it was shown that lactisole specifically inhibits human T1R3 but not the rodent form of the receptor (Sclafani, A, and Perez, C., Physiol Behav 1997, 61, 25-29; Schiffman, S. S., et al., Chem Senses 1999, 24, 439-447) Lactisole (by blocking hT1R3 subunit) is a broad acting inhibitor of all or most sweeteners, and a suspected inverse agonist of the sweet receptor that on wash-out produces a sweet after-taste in humans (Schiffman, supra; Bond, R. A., and Ijzerman, A. P., Trends Pharmacol Sci 2006, 27, 92-96; Galindo-Cuspinera, V., et al., Nature 2006, 441, 354-357). By blocking hT1R3, lactisole alos inhibits hT1R1+hT1R3. Compared to activities of agonists of T1R receptors, very little is known of physiological and medicinal roles for sweet and umami receptor antagonists.

The use of fibrates and phenoxy-herbicides in medicine and/or agriculture, respectively, is determined by their predominant activities. The fibrates are a class of amphipathic carboxylic acids with a phenoxy acid motif. Fibrates are used to treat hyperlipidemias: they bind and activate peroxisome proliferator-activated receptor alpha (PPAR-alpha) which affects lipid metabolism to lower triglycerides predominantly, along with a modest lowering of LDL and increase of HDL (Staels, B., and Fruchart, J. C., Diabetes 2005, 54, 2460-2470). Some fibrates also have an effect on glycemia and insulin resistance (Staels, supra; and Willson, T. M., et al., J Med Chem 2000, 43, 527-550).

Phenoxy herbicides are a class of organo-auxins used extensively in agriculture to control broad-leaf weeds (Troyer, J. R., Weed Science 2001, 49, 290-297). Approximately 55 million pounds of phenoxy herbicides are used annually in the United States, with 2,4-D comprising 86% of total use or about 47 million pounds of acid equivalent (Szmedra, P., Weed Science 1997, 45, 592-598).

Fibrates and phenoxy herbicides are structurally, and to some extent functionally, similar. For example one of the first widely used fibrates, clofibric acid, actually has demonstrated herbicidal activity (Lahey, K. A., et al., Mol Plant Microbe Interact 2004, 17, 1394-1401). Conversely, it has been shown that some phenoxy herbicides such as 2,4D (2,4-dichlorophenoxyacetic acid) and MCPA (4-chloro-2-methylphenoxyacetic acid) have fibrate-like effects that lower lipid levels in rats (Vainio, H., et al., Biochem Pharmacol 1983, 32, 2775-2779).

Considering that the aforementioned chemicals are intended for human use and/or consumption, it is important to better understand the mechanisms of action and to identify potential binding partners of these structurally similar compounds.

### SUMMARY OF THE INVENTION

The present disclosure relates to materials and methods for modulating the activity of human type I taste receptor subunit 3 (hT1R3). In one embodiment of the invention, a method of modulating the activity of human type I taste receptor subunit 3 (hT1R3) is provided comprising administering to a subject an effective amount of a modulator having a structure of formula (I):

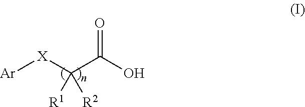

wherein Ar is an aryl group; R¹ and R² are each independently hydrogen or C₁₋₆alkyl; X is null or O; and n is 1, 2, 3, or 4, or a salt or ester thereof; with the proviso that when R¹ is methyl, R² is hydrogen, and X is O, Ar is not 4-methoxyphenyl; wherein the modulator binds to and modulates the activity of hT1R3.

In another embodiment, the aforementioned method is provided wherein Ar is selected from the group consisting of phenyl, naphthyl, and imidazoyl. In still another embodiment, the aforementioned method is provided wherein the phenyl, naphthyl, or imidazoyl is substituted with C₁₋₆alkyl, halo, or both. In yet another embodiment, the aforementioned method is provided wherein the halo is chloro. In another embodiment, an aforementioned method is provided wherein R¹ and R² are each hydrogen or methyl. In still another embodiment, the aforementioned method is provided wherein R¹ and R² are both methyl. In yet another embodiment, an aforementioned method is provided wherein X is O. Still another embodiment o the invention provides an aforementioned method wherein the modulator further activates peroxisome proliferators-activated receptor a. In still another embodiment, an aforementioned method is provided wherein the modulator further acts as an herbicide. In yet another embodiment, an aforementioned method is provided wherein the modulator is selected from the group consisting of:

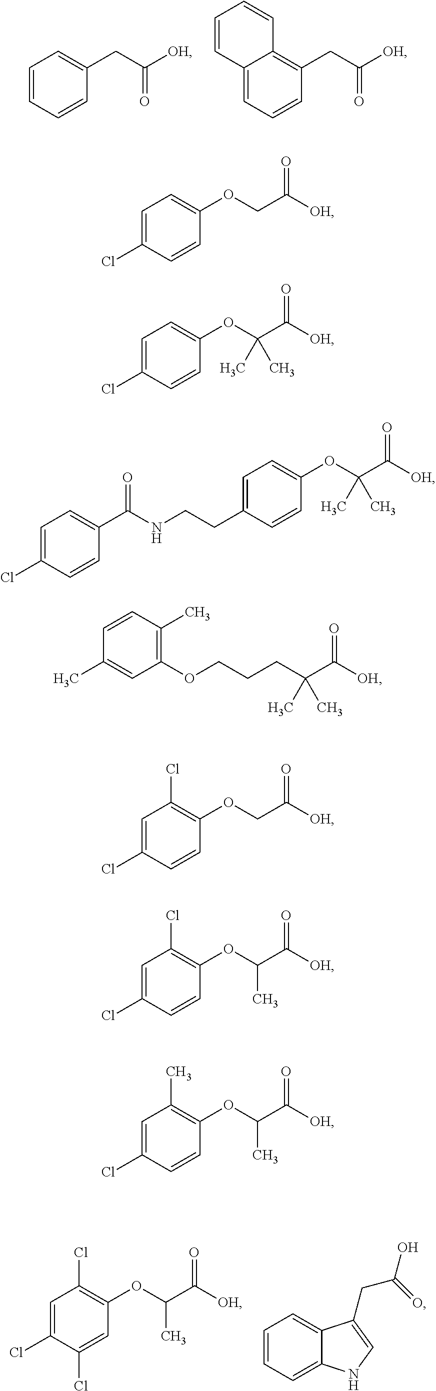

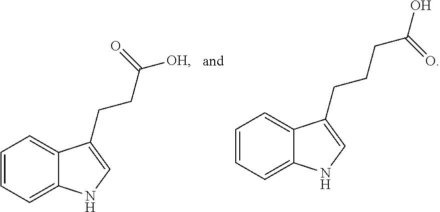

Methods of treating are also contemplated by the present invention. In one embodiment, a method of treating a disorder associated with lipid metabolism selected from the group consisting of: hyperlipidemia, atherosclerosis, acute pancreatitis, hypercholesterolemia, is provided, the method comprising administering to a subject an effective amount of a modulator having a structure of formula (I):

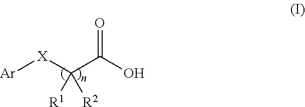

wherein Ar is an aryl group; R¹ and R² are each independently hydrogen or C₁₋₆alkyl; X is null or O; and n is 1, 2, 3, or 4, or a salt or ester thereof; with the proviso that when R¹ is methyl, R² is hydrogen, and X is O, Ar is not 4-methoxyphenyl; wherein the modulator binds to and modulates the activity of hT1R3, thereby treating a disorder associated with lipid metabolism.

In another embodiment, the aforementioned method is provided wherein Ar is selected from the group consisting of phenyl, naphthyl, and imidazoyl. In still another embodiment, the aforementioned method is provided wherein the phenyl, naphthyl, or imidazoyl is substituted with C₁₋₆alkyl, halo, or both. In yet another embodiment, the aforementioned method is provided wherein the halo is chloro. In another embodiment, an aforementioned method is provided wherein R¹ and R² are each hydrogen or methyl. In still another embodiment, the aforementioned method is provided wherein R¹ and R² are both methyl. In yet another embodiment, an aforementioned method is provided wherein X is O, Still another embodiment o the invention provides an aforementioned method wherein the modulator further activates peroxisome proliferators-activated receptor α. In still another embodiment, an aforementioned method is provided wherein the modulator further acts as an herbicide. In yet another embodiment, an aforementioned method is provided wherein the modulator is selected from the group consisting of:

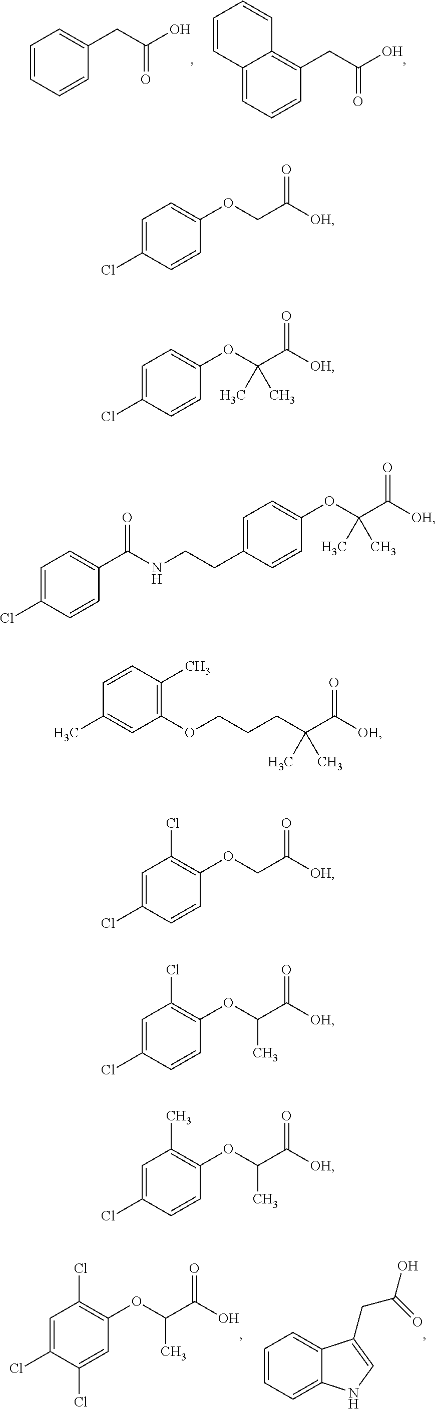

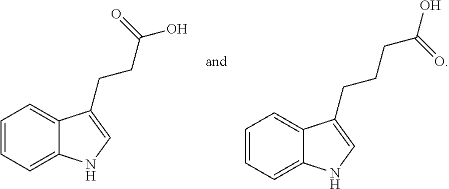

In another embodiment of the invention, a method of treating a disorder associated with carbohydrate metabolism selected from the group consisting of: obesity, metabolic syndrome, hyperglycemia, hypertriglyceridemia, diabetes type I, diabetes type II, and hypertension, is provided, the method comprising administering to a subject an effective amount of a modulator having a structure of formula (I):

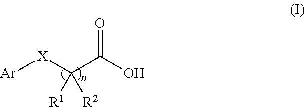

wherein Ar is an aryl group; R¹ and R² are each independently hydrogen or C₁₋₆alkyl; X is null or O; and n is 1, 2, 3, or 4, or a salt or ester thereof; with the proviso that when R¹ is methyl, R² is hydrogen, and X is O, Ar is not 4-methoxyphenyl; wherein the modulator binds to and modulates the activity of hT1R3, thereby treating a disorder associated with carbohydrate metabolism.

In another embodiment, the aforementioned method is provided wherein Ar is selected from the group consisting of phenyl, naphthyl, and imidazoyl. In still another embodiment, the aforementioned method is provided wherein the phenyl, naphthyl, or imidazoyl is substituted with C₁₋₆alkyl, halo, or both. In yet another embodiment, the aforementioned method is provided wherein the halo is chloro. In another embodiment, an aforementioned method is provided wherein R¹ and R² are each hydrogen or methyl. In still another embodiment, the aforementioned method is provided wherein R¹ and R² are both methyl. In yet another embodiment, an aforementioned method is provided wherein X is O, Still another embodiment o the invention provides an aforementioned method wherein the modulator further activates peroxisome proliferators-activated receptor α. In still another embodiment, an aforementioned method is provided wherein the modulator further acts as an herbicide. In yet another embodiment, an aforementioned method is provided wherein the modulator is selected from the group consisting of:

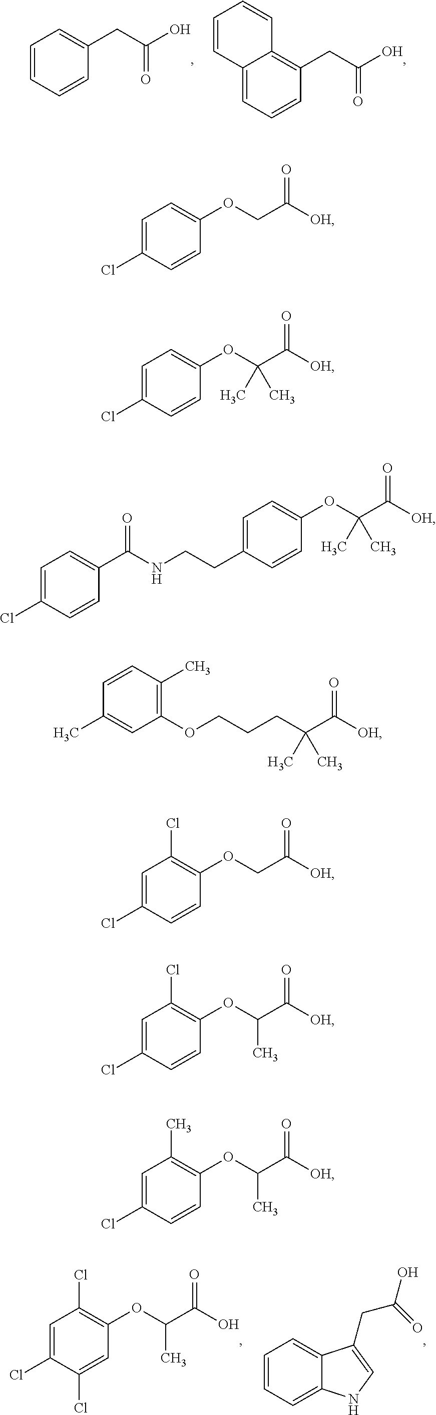

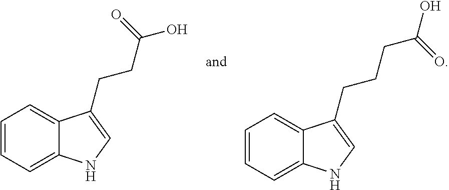

In still another embodiment, a method of treating a disorder associated with impaired carbohydrate absorption selected from the group consisting of: anorexia, bulimia, intestinal malabsorption syndromes, and celiac disease, is provided, the method comprising administering to a subject an effective amount of a modulator having a structure of formula (I):

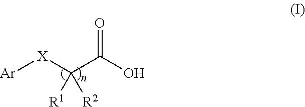

wherein Ar is an aryl group; R¹ and R² are each independently hydrogen or C₁₋₆alkyl; X is null or O; and n is 1, 2, 3, or 4, or a salt or ester thereof; with the proviso that when R¹ is methyl, R² is hydrogen, and X is O, Ar is not 4-methoxyphenyl; wherein the modulator binds to and modulates the activity of hT1R3, thereby treating a disorder associated with carbohydrate metabolism.

In another embodiment, the aforementioned method is provided wherein Ar is selected from the group consisting of phenyl, naphthyl, and imidazoyl. In still another embodiment, the aforementioned method is provided wherein the phenyl, naphthyl, or imidazoyl is substituted with C₁₋₆alkyl, halo, or both. In yet another embodiment, the aforementioned method is provided wherein the halo is chloro. In another embodiment, an aforementioned method is provided wherein R¹ and R² are each hydrogen or methyl. In still another embodiment, the aforementioned method is provided wherein R¹ and R² are both methyl. In yet another embodiment, an aforementioned method is provided wherein X is O, Still another embodiment o the invention provides an aforementioned method wherein the modulator further activates peroxisome proliferators-activated receptor α. In still another embodiment, an aforementioned method is provided wherein the modulator further acts as an herbicide. In yet another embodiment, an aforementioned method is provided wherein the modulator is selected from the group consisting of:

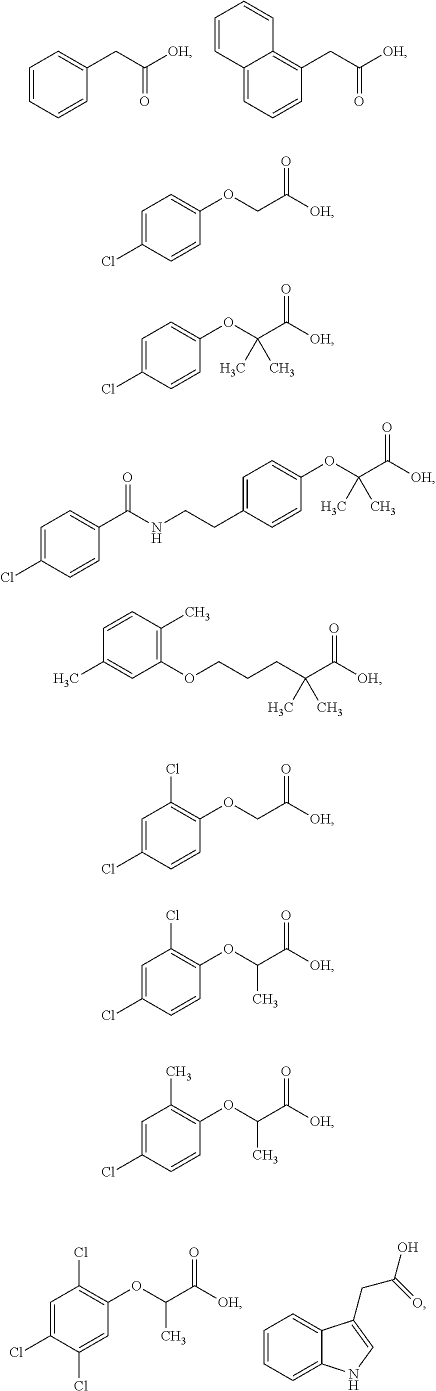

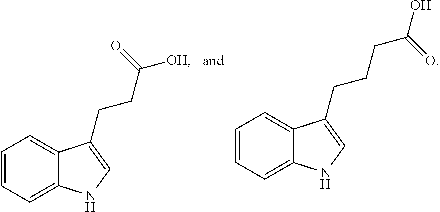

In related embodiments, an aforementioned is provided wherein the subject is a human, wherein the modulator is administered orally, and/or wherein the modulator is administered by injection.

In yet another embodiment of the invention, a method of screening for a modulator of hT1R3 activity is provided comprising the steps of: (a) contacting a cell expressing hT1R3 with a candidate compound having a structure of formula (I):

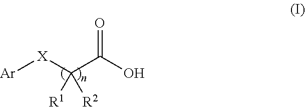

wherein Ar is an aryl group; R¹ and R² are each independently hydrogen or C₁₋₆alkyl; X is null or O; and n is 1, 2, 3, or 4, or a salt or ester thereof; with the proviso that when R¹ is methyl, R² is hydrogen, and X is O, Ar is not 4-methoxyphenyl; (b) measuring the activity of hT1R3; (c) comparing the activity of hT1R3 measured in step (b) to the activity of a hT1R3 in the absence of the candidate compound; and (d) identifying the candidate compound as a modulator of hT1R3 activity if an increase or decrease in hT1R3 activity is measured relative to the absence of said candidate compound.

In another embodiment, the aforementioned method is provided wherein Ar is selected from the group consisting of phenyl, naphthyl, and imidazoyl. In still another embodiment, the aforementioned method is provided wherein the phenyl, naphthyl, or imidazoyl is substituted with C₁₋₆alkyl, halo, or both. In yet another embodiment, the aforementioned method is provided wherein the halo is chloro. In another embodiment, an aforementioned method is provided wherein R¹ and R² are each hydrogen or methyl. In still another embodiment, the aforementioned method is provided wherein R¹ and R² are both methyl. In yet another embodiment, an aforementioned method is provided wherein X is O. Still another embodiment o the invention provides an aforementioned method wherein the modulator further activates peroxisome proliferators-activated receptor α. In still another embodiment, an aforementioned method is provided wherein the modulator further acts as an herbicide. In yet another embodiment, an aforementioned method is provided wherein the modulator is selected from the group consisting of:

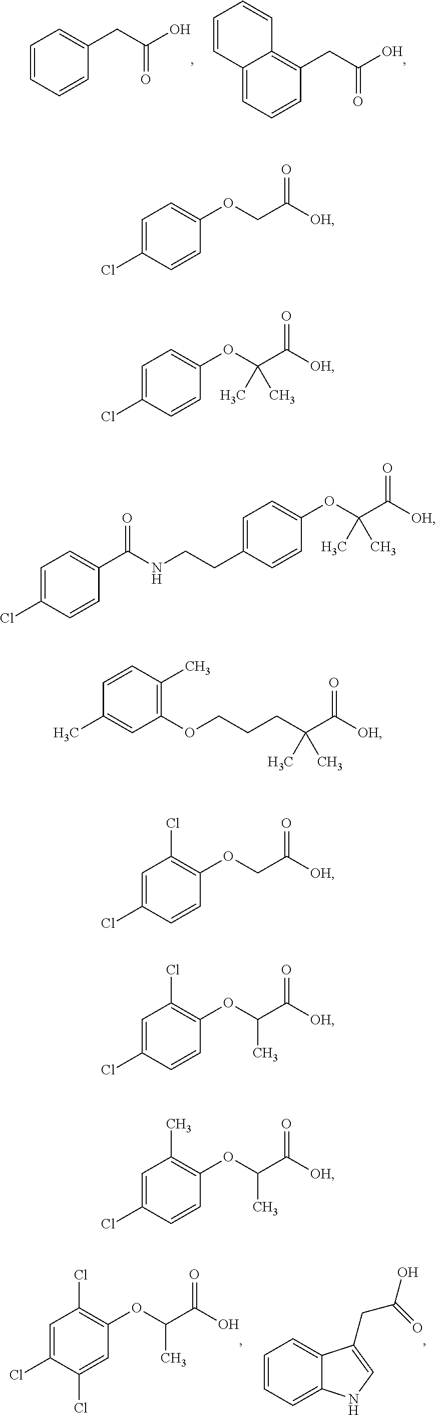

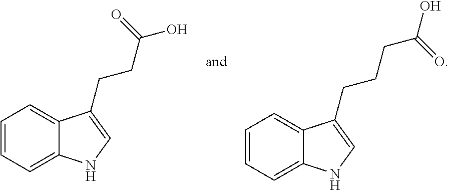

In yet another embodiment of the invention, an aforementioned is provided wherein the contacting occurs in vivo. In another embodiment, the contacting occurs in a rodent expressing hT1R3.

In still another embodiment of the invention, a modulator of T1R3 is provided having a structure of formula (I):

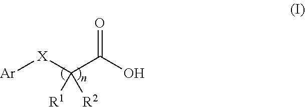

wherein Ar is an aryl group; R¹ and R² are each independently hydrogen or C₁₋₆alkyl; X is null or O; and n is 1, 2, 3, or 4, or a salt or ester thereof; with the proviso that the modulator is not a compound selected from the group consisting of:

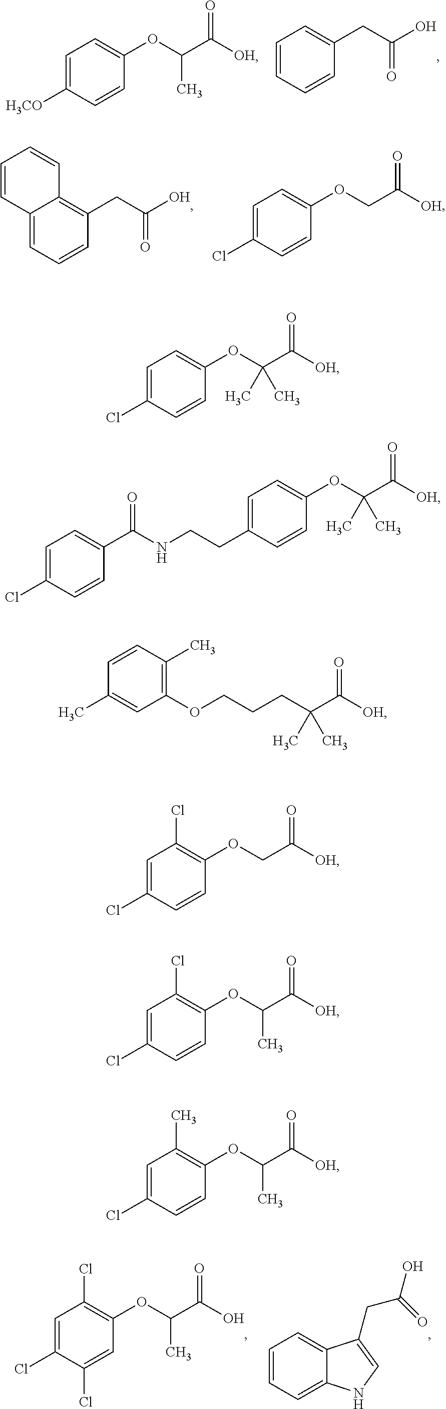

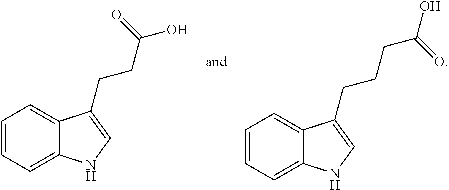

### BRIEF DESCRIPTION OF THE DRAWING

The following drawing forms part of the present specification and is included to further illustrate aspects of the present disclosure. The disclosure may be better understood by reference to the drawing in combination with the detailed description of the specific embodiments presented herein.

FIG. 1 shows the chemical structures of lactisole, phenoxy herbicides and fibrates.

FIG. 2 shows that phenoxy herbicides and fibrates potently inhibit the human sweet-sensing receptor T1R2+T1R3. T1R2+T1R3 subunits were transfected into HEK cells along with the promiscuous Gq-type protein G16-gust44. Activation of receptors by agonists was followed using the calcium-sensitive fluorescent dye fluo-4-AM in a 96-well format with the fluorescent microplate reader Flexstation II (Molecular Devices). Inhibitors were added concomitantly with the sweetener sucralose at 2.5 mM (saturating concentration). FIG. 2A. Example of time-resolved raw traces of sucralose-activated T1R2+T1R3 receptor calcium response in the presence of increasing concentrations of the lactisole (10 to 100 μM). Fluorescence signal (F) is expressed in arbitrary units (AU). The down arrow indicates the injection of the sweetener-inhibitors mixture on the cells. FIG. 2B, FIG. 2C. Dose-response curves showing inhibition of sucralose-activated human sweet receptor T1R2+T1R3 by phenoxy-herbicides and fibrates. Peak calcium signal is expressed as ΔF/F in %, normalized to control (sucralose alone). Data are from a representative experiment, means±SD, done in quadruplicate.

FIG. 3 shows human T1R3 transmembrane domain determines sensitivity to lactisole, phenoxy-herbicides and fibrates. Human (hT1R2+hT1R3), mouse (mT1R2+mT1R3) and chimeric mouse/human sweet receptors (as indicated by m/h prefix) were activated by 2.5 mM sucralose and assayed for inhibition by indicated phenoxy-herbicides, fibrates and lactisole at sub maximal concentrations (˜5-times IC50). Chimeric receptors of T1R3 contained the extracellular portion (VFTM and CRD) of human or mouse receptor, and the transmembrane domain and C-terminal from mouse or human receptor (h.1-567.mT1R3 and m.1-568.hT1R3, respectively). Inhibition of the sucralose response occurred in the presence of the transmembrane domain of human T1R3. Data in the figure are from a representative experiment, means±SD, done in quadruplicate.

FIG. 4 shows human T1R3 cDNA

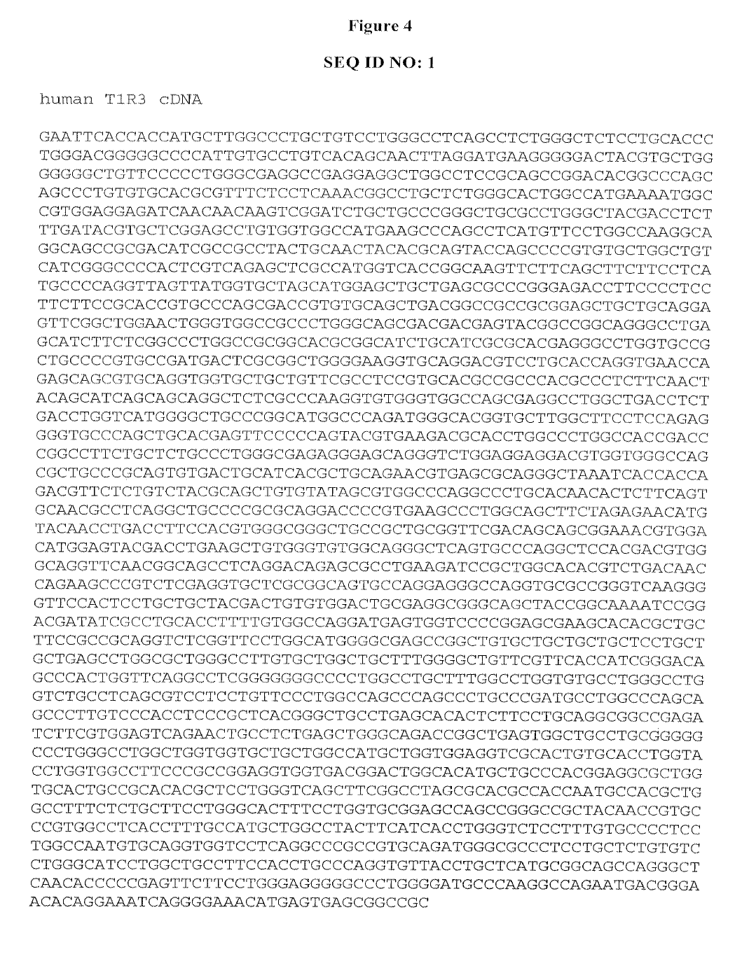

FIG. 5 shows human T1R3

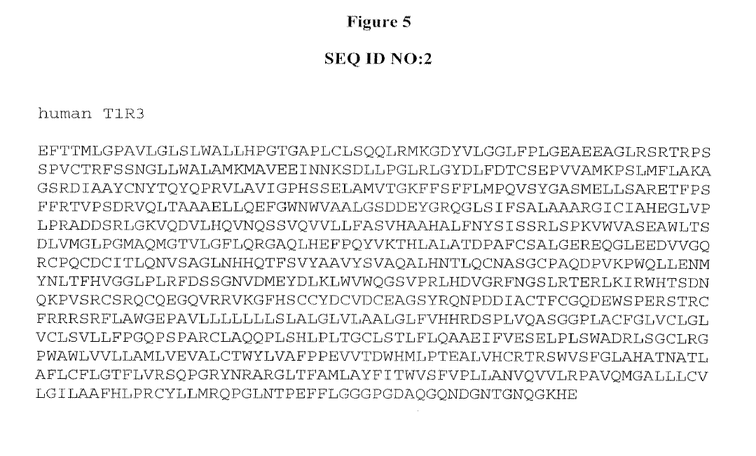

FIG. 6 shows Mouse T1R3 cDNA

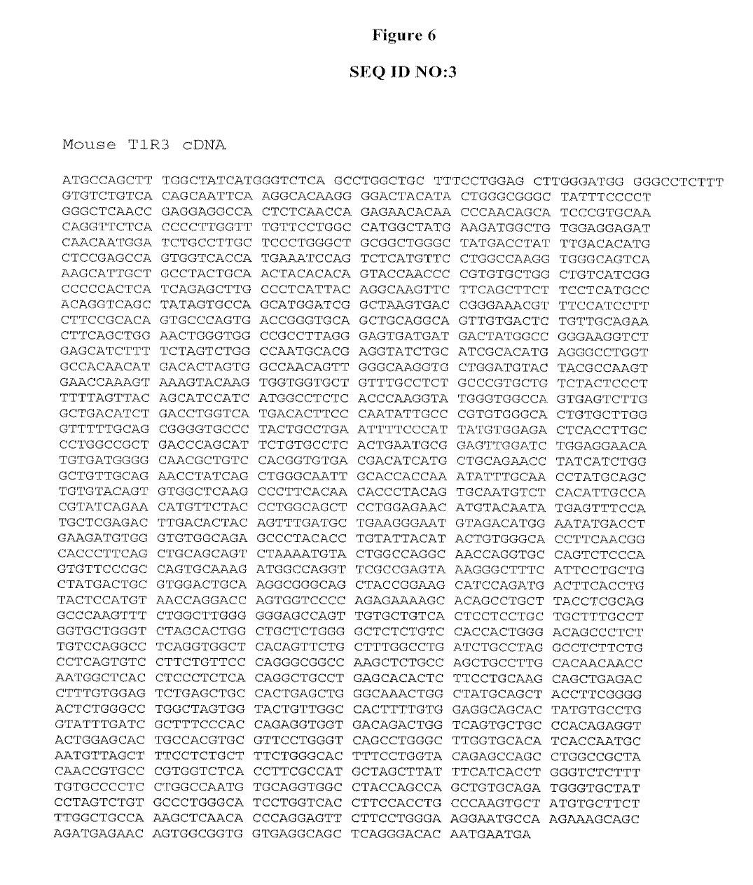

FIG. 7 shows Mouse T1R3

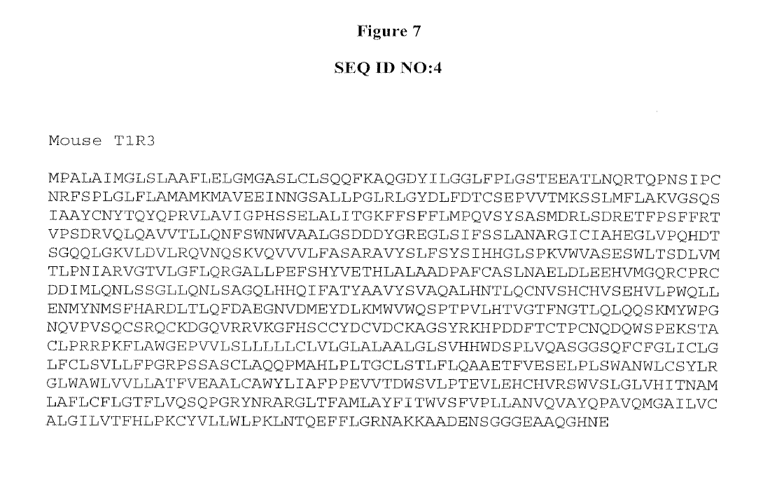

FIG. 8 shows expression of T1R3-GFP in testis of transgenic mice. Upper panel 10×, lower panel 20× magnification.

FIG. 9 shows expression of T1R3-GFP in gut of transgenic mice. Confocal images. 63× magnification.

FIG. 10 shows expression of T1R3-GFP in retina of transgenic mice. CRI (Cambridge Research & Instrumentation, Inc., Woburn, Mass.) multispectral imaging technique, image shown is after removal of background autofluorescence. 10× magnification.

### DETAILED DESCRIPTION OF THE PREFERRED EMBODIMENTS OF THE INVENTION

The present invention describes the inhibition of human but not rodent T1R3 by phenoxy-auxin herbicides and lipid-lowering fibrates. T1R3 as a co-receptor in taste cells responds to sweet compounds and amino-acids; in endocrine cells of gut and pancreas, T1R3 contributes to glucose sensing. Thus, certain effects of fibrates in treating hyperlipidemia and type II diabetes may be realized via actions on T1R3. Likewise, phenoxy-herbicides may have adverse metabolic effects in humans that would have gone undetected in studies on rodents. Accordingly, the disclosure provides modulator compounds and methods of treating a variety of carbohydrate or lipid metabolic disorders.

A “sweet tastant”, as defined herein, is a compound or molecular complex that induces, in a subject, the perception of a sweet taste. In particular, a sweet tastant is one which results in the activation of the T1R3 protein resulting in one or more of the following: (i) an influx of Ca⁺² into the cell; (ii) release of Ca⁺² from internal stores; (iii) activation of coupled G proteins such as Gs and/or gustducin; (iv) modulation of second messenger-regulating enzymes such as adenylyl cyclase and/or phospholipase C. Examples of sweet tastants include but are not limited to saccharin or sucrose, or other sweeteners.

The terms “pharmaceutically” or “pharmacologically acceptable” refer to molecular entities and compositions that are stable, inhibit protein degradation such as aggregation and cleavage products, and in addition do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like, including those agents disclosed above.

As used herein, “therapeutically effective amount” is a dose resulting in a desirable change or treatment in a mammal having a disorder as described herein.

Unless otherwise defined, all technical and scientific terms used herein have the same meaning as commonly understood by one of ordinary skill in the art to which the technology disclosed herein belongs. The following references provide one of skill with a general definition of many of the terms used in this disclosure: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that, as used in this specification and the appended claims, the singular forms “a,” “an,” and “the” include plural reference unless the context clearly dictates otherwise.

T1R3

Members of the T1R family of taste-cell-specific GPCRs are identified in Hoon et al., Cell, 96:541-551 (1999), WO 00/06592, and WO 00/06593. US Publication No. 2009/0217391 describes human T1R3. All of the aforementioned disclosures are incorporated herein by reference in their entireties.

A. T1R3 Polynucleotides

The cDNA sequence and deduced amino acid sequence of human T1R3 is set out in SEQ ID NOs: 1 and 2, respectively. The T1R3 nucleotide sequences of the invention include: (a) the DNA sequence shown in SEQ ID NO: 1; (b) nucleotide sequences that encode the amino acid sequence shown in SEQ ID NO:2; (c) any nucleotide sequence that (i) hybridizes to the nucleotide sequence set forth in (a) or (b) under stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3) and (ii) encodes a functionally equivalent gene product; and (d) any nucleotide sequence that hybridizes to a DNA sequence that encodes the amino acid sequence shown in SEQ ID NO:2, under less stringent conditions, such as moderately stringent conditions, e.g., washing in 0.2×SSC/0.1% SDS at 42° C. (Ausubel et al., 1989 supra), yet which still encodes a functionally equivalent T1R3 gene product. Functional equivalents of the T1R3 protein include naturally occurring T1R3 present in species other than humans. The invention also includes degenerate variants of sequences (a) through (d). The invention also includes nucleic acid molecules, that may encode or act as T1R3 antisense molecules, useful, for example, in T1R3 gene regulation (forward and/or as antisense primers in amplification reactions of T1R3 gene nucleic acid sequences).

In addition to the T1R3 nucleotide sequences described above, homologs of the T1R3 gene present in other species can be identified and readily isolated, without undue experimentation, by molecular biological techniques well known in the art. For example, cDNA libraries, or genomic DNA libraries derived from the organism of interest can be screened by hybridization using the nucleotides described herein as hybridization or amplification probes, preferably using stringent hybridization conditions such as the conditions described above.

The invention also encompasses nucleotide sequences that encode mutant T1R3s, peptide fragments of T1R3, truncated T1R3, and T1R3 fusion proteins. These include, but are not limited to, nucleotide sequences encoding polypeptides or peptides corresponding to functional domains of T1R3, including but not limited to, the ATD (amino terminal domain) that is believed to be involved in ligand binding and dimerization, the cysteine rich domain, and/or the transmembrane spanning domains of T1R3, or portions of these domains; truncated T1R3s in which one or two domains of T1R3 are deleted, e.g., a functional T1R3 lacking all or a portion of the ATD region. Nucleotides encoding fusion proteins may include, but are not limited to, full-length T1R3, truncated T1R3 or peptide fragments of T1R3 fused to an unrelated protein or peptide such as an enzyme, fluorescent protein, luminescent protein, and the like, which can be used as a marker.

Based on the model of T1R3's structure, it is predicted that T1R3 dimerizes to form a functional receptor. Thus, certain of these truncated or mutant T1R3 proteins may act as dominant-negative inhibitors of the native T1R3 protein.

T1R3 nucleotide sequences may be isolated using a variety of different methods known to those skilled in the art. For example, a cDNA library constructed using RNA from a tissue known to express T1R3 can be screened using a labeled T1R3 probe. Alternatively, a genomic library may be screened to identify nucleic acid molecules encoding the T1R3 receptor protein. Further, T1R3 nucleic acid sequences may be derived by performing PCR using two oligonucleotide primers designed on the basis of the T1R3 nucleotide sequences disclosed herein. The template for the reaction may be cDNA obtained by reverse transcription of mRNA prepared from cell lines or tissue known to express T1R3.

The invention also encompasses (a) DNA vectors that contain any of the foregoing T1R3 sequences and/or their complements (i.e., antisense); (b) DNA expression vectors that contain any of the foregoing T1R3 sequences operatively associated with a regulatory element that directs the expression of the T1R3 coding sequences; (c) genetically engineered host cells that contain any of the foregoing T1R3 sequences operatively associated with a regulatory element that directs the expression of the T1R3 coding sequences in the host cell; and (d) transgenic mice or other organisms that contain any of the foregoing T1R3 sequences. As used herein, regulatory elements include, but are not limited to, inducible and non-inducible promoters, enhancers, operators and other elements known to those skilled in the art that drive and regulate expression.

B. T1R3 Polypeptides

T1R3 protein, polypeptides and peptide fragments, mutated, truncated or deleted forms of the T1R3 and/or T1R3 fusion proteins can be prepared for a variety of uses, including but not limited to the generation of antibodies, the identification of other cellular gene products involved in the regulation of T1R3-mediated taste transduction, and the screening for compounds that can be used to modulate taste perception such as novel sweeteners and taste modifiers as well as therapeutics useful in or the treatment of carbohydrate or lipid metabolic disorders.

As used herein a “polypeptide” refers to a polymer composed of amino acid residues, structural variants, related naturally occurring structural variants, and synthetic non-naturally occurring analogs thereof linked via peptide bonds. Synthetic polypeptides can be prepared, for example, using an automated polypeptide synthesizer. The term “protein” typically refers to large polypeptides, as well as proteinaceous forms comprising a plurality of polypeptide or peptide chains. The term “peptide” typically refers to short polypeptides.

As used herein a “fragment” of a polypeptide is meant to refer to any portion of a polypeptide or protein smaller than the full-length polypeptide or protein expression product.

SEQ ID NO:2 shows the amino acid sequence of the human T1R3 protein deduced from the cognate polynucleotide sequence set forth in SEQ ID NO:1. The T1R3 amino acid sequences of the disclosure include the amino acid sequence shown in SEQ ID NO:2. Further, T1R3s of other species are encompassed by the disclosure. In fact, any T1R3 protein encoded by the T1R3 nucleotide sequences described herein is within the scope of the claimed subject matter.

The disclosure also contemplates proteins that are functionally equivalent to the T1R3 encoded by the nucleotide sequences described herein, as judged by any of a number of criteria, including but not limited to the ability of a sweet tastant to activate T1R3 in a taste receptor cell, leading to transmitter release from the taste receptor cell into the synapse and activation of an afferent nerve. Such functionally equivalent T1R3 proteins include but are not limited to proteins having additions or substitutions of amino acid residues within the amino acid sequence encoded by the T1R3 nucleotide sequences described herein but which result in a protein retaining a detectable level of activity up to and including full activity, i.e., functionally a silent change, thus producing a functionally equivalent gene product.

Polypeptides corresponding to one or more domains of T1R3 (e.g., amino-terminal domain, the cysteine-rich domain and/or the transmembrane spanning domains), truncated or deleted T1R3s (e.g., T1R3 in which the amino terminal domain, the cysteine-rich domain and/or the transmembrane spanning domains are deleted) as well as fusion proteins in which the full-length T1R3, a T1R3 peptide or a truncated T1R3 is fused to an unrelated protein are also within the scope of the disclosure and can be designed on the basis of the T1R3 nucleotide and T1R3 amino acid sequences disclosed herein. Such fusion proteins include fusions to an enzyme, fluorescent protein, or luminescent protein which provide a marker function.

While the T1R3 polypeptides and peptides can be chemically synthesized (see, e.g., Creighton, 1983, Proteins: Structures and Molecular Principles, W. H. Freeman & Co., N.Y.), large polypeptides derived from T1R3 and the full-length T1R3 itself may be advantageously produced by recombinant DNA technology using techniques well known in the art for expressing a nucleic acid containing T1R3 gene sequences and/or coding sequences. Such methods can be used to construct expression vectors containing the T1R3 nucleotide sequences described herein and appropriate transcriptional and translational control signals. These methods include, for example, in vitro recombinant DNA techniques, synthetic techniques, and in vivo genetic recombination. (See, for example, the techniques described in Sambrook et al., 1989, supra, and Ausubel et al., 1989, supra.)

A variety of host-expression vector systems may be utilized to express the T1R3 nucleotide sequences of the disclosure. Where the T1R3 peptide or polypeptide is expressed as a soluble derivative (e.g, peptides corresponding to the amino-terminal domain the cysteine-rich domain and/or the transmembrane spanning domain) and is not secreted, the peptide or polypeptide can be recovered from the host cell. Alternatively, where the T1R3 peptide or polypeptide is secreted, the peptide or polypeptides may be recovered from the culture media. However, the expression systems also include engineered host cells that express T1R3 or functional equivalents, anchored in the cell membrane. Purification or enrichment of the T1R3 from such expression systems can be accomplished using appropriate detergents, lipid micelles, and methods well known to those skilled in the art. Such engineered host cells themselves may be used in situations where it is important not only to retain the structural and functional characteristics of the T1R3, but to assess biological activity, i.e., in drug screening assays.

The expression systems that may be used for purposes of the invention include but are not limited to microorganisms such as bacteria transformed with recombinant bacteriophage, plasmid or cosmid DNA-expression vectors containing T1R3 nucleotide sequences; yeast transformed with recombinant yeast expression vectors containing T1R3 nucleotide sequences or mammalian cell systems harboring recombinant expression constructs containing promoters derived from the genome of mammalian cells or from mammalian viruses.

Appropriate expression systems can be chosen to ensure that the correct modification, processing and sub-cellular localization of the T1R3 protein occurs. To this end, eukaryotic host cells which possess the ability to properly modify and process the T1R3 protein are preferred. For long-term, high yield production of recombinant T1R3 protein, such as that desired for development of cell lines for screening purposes, stable expression is preferred. Rather than using expression vectors which contain origins of replication, host cells can be transformed with DNA controlled by appropriate expression control elements and a selectable marker gene, i.e., tk, hgprt, dhfr, neo, or hygro. Following the introduction of the foreign DNA, engineered cells may be allowed to grow for 1-2 days in enriched media, and then switched to a selective media. Such engineered cell lines are expected to be particularly useful in screening and evaluation of compounds that modulate the endogenous activity of the T1R3 gene product.

C. Transgenic and Knock-in Animals

The T1R3 gene products can also be expressed in transgenic or knock-in animals. Animals of any species, including, but not limited to, mice, rats, rabbits, guinea pigs, pigs, micro-pigs, goats, and non-human primates, e.g., baboons, monkeys, and chimpanzees may be used to generate T1R3 transgenic animals.

Any technique known in the art may be used to introduce the T1R3 transgene into animals to produce the founder lines of transgenic animals. Such techniques include, but are not limited to, pronuclear microinjection (Hoppe, P. C. and Wagner, T. E., 1989, U.S. Pat. No. 4,873,191); retrovirus-mediated gene transfer into germ lines (van der Putten et al., 1985, Proc Natl Acad Sci USA 82:6148-6152); gene-targeting in embryonic stem cells (Thompson et al., 1989, Cell, 56:313-321); electroporation of embryos (Lo, 1983, Mol Cell Biol 3:1803-1814); and sperm-mediated gene transfer (Lavitrano et al., 1989, Cell 57:717-723); etc. For a review of such techniques, see Gordon, 1989, Transgenic Animals, Intl Rev Cytol 115:171-229, which is incorporated by reference herein in its entirety.

The present disclosure provides for transgenic animals that carry the HUMAN T1R3 transgene in all their cells, as well as animals which carry the transgene in some, but not all, their cells, i.e., mosaic animals. The transgene may also be selectively introduced into and activated in a particular cell type by following, for example, the teaching of Lasko et al., (Lasko, M. et al., 1992, Proc Natl Acad Sci USA 89:6232-6236). The regulatory sequences required for such a cell-type specific activation will depend upon the particular cell type of interest, and will be apparent to those of skill in the art. When it is desired that the T1R3 transgene be integrated into the chromosomal site of the endogenous T1R3 gene, gene-targeting is preferred. Briefly, when such a technique is to be utilized, vectors containing some nucleotide sequences homologous to the endogenous T1R3 gene are designed for the purpose of integrating, via homologous recombination with chromosomal sequences, into and disrupting the function of the nucleotide sequence of the endogenous T1R3 gene (i.e., insertional inactivation)

Once transgenic animals have been generated, the expression of the recombinant T1R3 gene may be assayed utilizing standard techniques. Initial screening may be accomplished by Southern blot analysis or PCR techniques to analyze animal tissues to determine whether integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and RT-PCR. Samples of T1R3 gene-expressing tissue may also be evaluated immunocytochemically using antibodies specific for the T1R3 transgene product.

Methods for generating gene “knock-in” animals are known in the art (Castrop H., (2010) Pflugers Arch 459(4):557-67; Chaible L M, et al., (2010) Genet Mol Res 9(3):1469-82; Arakawa H, and Buerstedde JM. (2006) Subcell Biochem 40:1-9; and Guan C, et al., (2010) Genesis 48(2):73-85.) In this procedure the endogenous gene is replaced by an in vitro generated DNA construct. Because the new DNA construct is inserted inside of the locus that originally controls expression of the endogenous gene, the new construct is controlled in exactly the same way. Therefore expression of the construct exactly matches that of the endogenous gene.

Modulators of T1R3 Activity

As described herein, lactisole is a sweet taste inhibitor whose site of action has been mapped to the transmembrane domain of human T1R3 (Xu, supra, Jiang, supra, and Winnig, M., et al., BMC Neurosci 2005, 6, 22). Lactisole is a broad-acting inhibitor of all or most sweeteners, and is a suspected inverse agonist of the sweet receptor that on wash-out produces a sweet after-taste in humans.

Disclosed herein are compounds which modulate the T1R3 receptor. Accordingly, the disclosure provides modulator compounds and methods of treating a variety of carbohydrate or lipid metabolic disorders. Specifically, disclosed herein are compounds having a structure of formula (I):

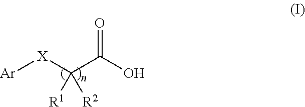

wherein Ar is an aryl or heteroaryl group; R¹ and R² are each independently hydrogen or C₁₋₆alkyl; X is null or O; and n is 1, 2, 3, or 4,
with the proviso that when R¹ is methyl, R² is hydrogen, and X is O, Ar is not 4-methoxyphenyl (i.e., lactisole).

As used herein, the term “alkyl” refers to straight-chained and branched hydrocarbon groups, nonlimiting examples of which include methyl, ethyl, and straight chain and branched propyl and butyl groups. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, ester, carboxylic acid, amide, guanidine, and amino groups.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one, two, three, or four groups independently selected from, for example, halo, alkyl, alkenyl, OCF₃, NO₂, CN, NC, OH, alkoxy, NH₂, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

Specific examples of compounds of formula (I) include, but are not limited to:

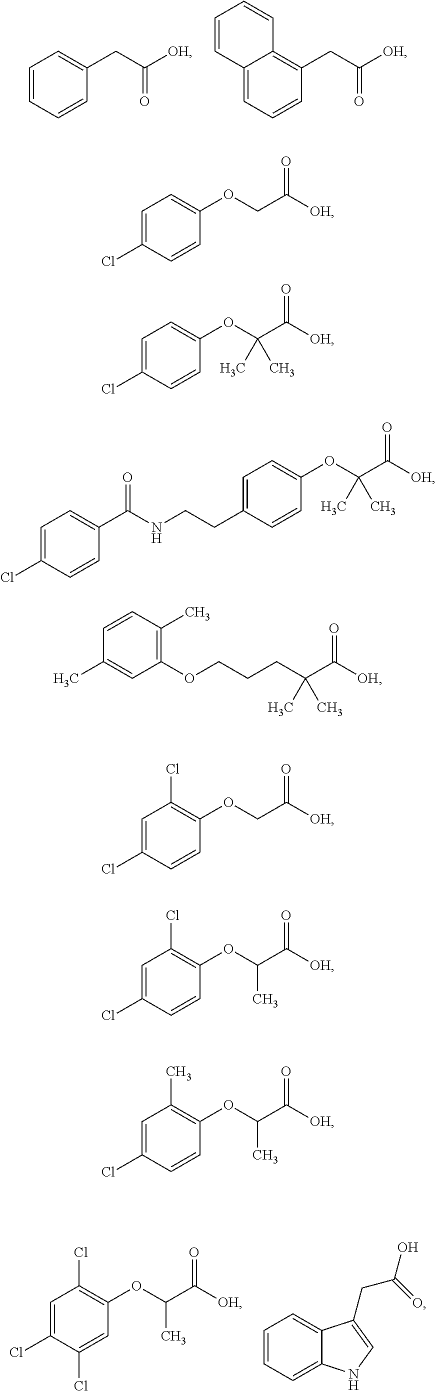

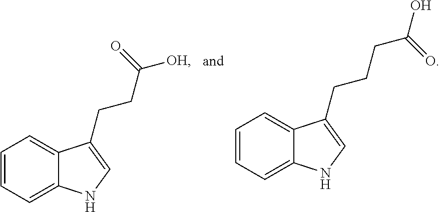

A. Herbicides

In some cases, the compounds of formula (I) also exhibit herbicide properties. Phenoxy-auxin herbicides are synthetic herbicides that mimic the action of auxin plant hormones. These herbicides are effective in killing broadleaf plants while leaving grasses largely unaffected, and are thus extensively used in crop agriculture and in landscape turf management (Szmedra, P., Weed Science 1997, 45, 592-598). Several of the phenoxy-herbicides described herein are among the most widely used, e.g. 2,4D. They have low soil sorption, high leachability, and are prone to enter the human food chain. Long-term biological effects of these compounds in humans are largely unknown and based on our studies their actions on T1R3-containing receptors would not have been apparent in rodent models.

B. Fibrates

In some cases, the compounds of formula (I) also activates peroxisome proliferator activated receptor-α (PPAR-α). Fibrates are used in the treatment of many forms of hyperlipidemia: these compounds primarily lower triglyceride levels, modestly improve HDL and seem to improve insulin resistance when the dyslipidemia is associated with other features of the metabolic syndrome (hypertension and diabetes mellitus type 2) (Steiner, G., Diab Vasc Dis Res 2007, 4, 368-374). The therapeutic target of fibrates is thought to be nuclear peroxisome proliferator activated receptor-alpha (PPAR-α), whose activation leads to increased transcription of several genes involved in lipid metabolism (Staels, B., and Fruchart, J. C., Diabetes 2005, 54, 2460-2470). Studies with PPAR agonists also reported lower plasma glucose, improved glucose tolerance, and enhanced insulin sensitivity (Staels, supra), although the mechanism of action of fibrates on glucose homeostasis remains unclear.

Diseases and Disorders Amenable to Treatment Using hT1R3 Modulators

The disclosure provides modulator compounds and methods of treating a variety of carbohydrate or lipid metabolic disorders. Diseases and/or disorders associated with carbohydrate metabolism include, but are not limited to, obesity, hypertension, metabolic syndrome, hyperglycemia, hypertriglyceridemia, diabetes type I, diabetes type II, as well as diabetic retinopathy and neuropathy, and other complications of diabetes, affecting many organs, such as skin, feet, heart, kidneys and including urological and sexual problems.

Assays to determine the therapeutic efficacy of candidate modulator compounds for the treatment of such diseases and/or disorders are known in the art. By way of example, therapeutic efficacy can be assayed by: monitoring fasting and non-fasting glycemia (e.g., acute test; mesurement of glucose content in a drop of blood); Glucose tolerance tests (e.g., Oral (OGTT): Patient receives 1.75 g glucose per kg of weight and blood samples are taken before the test and every 30 minutes after drinking the solution; and/or parenteral (IVGTT): Used to measure insulin response without the confounds of intestinal absorption. Patient receives i.v. injection glucose and blood samples are taken at 1,3, 5 min, respectively, after injection.); and monitoring blood parameters (e.g., including glucose, insulin, cholesterol, leptine, adiponectine, triglycerides, free fatty acids, ketones, glycerol; and lipid profile., including HDL, LDL and the like, as well as C-reactive protein, and glycosylated hemoglobin A1c test).

Diseases and/or disorders associated with lipid metabolism include, but are not limited to, hyperlipidemia, atherosclerosis, acute pancreatitis, and hypercholesterolemia.

In addition, hyperinsulinemia may be a causative factor in the development of high blood pressure, high levels of circulating low density lipo-proteins (LDLs), and lower than normal levels of the beneficial high density lipo-proteins (HDLs). While moderate insulin resistance can be compensated for in the early stages of Type II diabetes by increased insulin secretion, in advanced disease states insulin secretion is also impaired. Treatments of Type II diabetes preferably address both insulin resistance and faulty insulin secretion.

Insulin resistance and hyperinsulinemia have also been linked with two other metabolic disorders that pose considerable health risks: impaired glucose tolerance and metabolic obesity. Impaired glucose tolerance is characterized by normal glucose levels before eating, with a tendency toward elevated levels (hyperglycemia) following a meal. According to the World Health Organization, approximately 11% of the U.S. population between the ages of 20 and 74 are estimated to have impaired glucose tolerance. These individuals are considered to be at higher risk for diabetes and coronary artery disease.

Obesity may also be associated with insulin resistance. Some researchers believe that impaired glucose tolerance and diabetes are clinically observed and diagnosed only later in the disease process after a person has developed insulin resistance and hyperinsulinomia.

Assays to determine the therapeutic efficacy of candidate modulator compounds for the treatment of such diseases and/or disorders are known in the art (Maxwell Quick Medical Reference, Medline Plus Encyclopedia, American Medical Association Complete Medical Encyclopedia (American Medical Association (AMA) Complete Medical Encyclopedia), Clinical Chemistry eBook (Vol. 55, No. 7, JULY 2009).

Methods of Identifying Modulators of T1R3

The subject matter of the disclosure relates to screening assay systems designed to identify compounds or compositions that modulate human T1R3 activity (i.e., “hT1R3 modulators”) or T1R3 gene expression and, thus, may be useful for modulation of sweet taste perception. In one embodiment, the compounds are structurally similar to the herbicides and/or fibrates described herein.

In accordance with the invention, a cell-based assay system can be used to screen for compounds that modulate the activity of the T1R3 and, thereby, modulate the perception of sweetness. To this end, cells that endogenously express T1R3 can be used to screen for compounds. Alternatively, cell lines, such as 293 cells, COS cells, CHO cells, fibroblasts, and the like, genetically engineered to express T1R3, can be used for screening purposes. Preferably, host cells genetically engineered to express a functional T1R3 are those that respond to activation by sweet tastants, such as taste receptor cells. Further, ooyctes or liposomes engineered to express T1R3 may be used in assays developed to identify modulators of T1R3 activity.

In one embodiment, immortalized human (or primate) pancreatic beta and/or alpha cells are used in a cell-based assay (Labriola L, et al., BMC Cell Biol., 2009, 19; 10:49. Similarly any human, primate or old-wold monkey (e.g. Rhesus monkey) endocrine and enteroendocrine cells that express T1r3 would be an excellent system is also contemplated by the present disclosure. By way of example, hormonal output is measured in such assays.

In still another embodiment of the disclosure, a transgenic mouse that expresses human T1R3 is contemplated. In yet another embodiment, a knock-in mouse model is contemplated.

The disclosure provides for methods for identifying a compound that induces the perception of a sweet taste (a “sweetness activator” or “agonist”) comprising (i) contacting a cell expressing the T1R3 receptor with a test compound and measuring the level of T1R3 activation; (ii) in a separate experiment, contacting a cell expressing the T1R3 receptor protein with a vehicle control and measuring the level of T1R3 activation where the conditions are essentially the same as in part (i), and (iii) comparing the level of activation of T1R3 measured in part (i) with the level of activation of T1R3 in part (ii), wherein an increased level of activated T1R3 in the presence of the test compound indicates that the test compound is a T1R3 activator.

The disclosure also provides for methods for identifying a compound that inhibits the perception of a sweet taste (a “sweetness inhibitor” or “antagonist”) comprising (i) contacting a cell expressing the T1R3 receptor protein with a test compound in the presence of a sweet tastant and measuring the level of T1R3 activation; (ii) in a separate experiment, contacting a cell expressing the T1R3 receptor protein with a sweet tastant and measuring the level of T1R3 activation, where the conditions are essentially the same as in part (i) and (iii) comparing the level of activation of T1R3 measured in part (i) with the level of activation of T1R3 in part (ii), wherein a decreased level of activation of T1R3 in the presence of the test compound indicates that the test compound is a T1R3 inhibitor.

In various embodiments of the invention, T1R3 mutants are used in the screening methods. For example, T1R3 allelic variants (Chen, Q. Y., et al., Am J Clin Nutr. 2009 September; 90(3):770S-779S; and Kim, U. K., et al., Chem. Senses. 2006 September; 31(7):599-611) or different level of T1R3 expression (Fushan, A. A., et al., Curr Biol. 2009 Aug. 11; 19(15):1288-93) can affect how the inhibitors would block the receptor. Such receptor blocking can be tested in vitro and in vivo in those animal models described herein.

In assays for modulators of hT1R3 activity, the cells expressing the T1R3 receptor are exposed to a test compound or to vehicle controls e.g., placebos. After exposure, the cells can be assayed to measure the expression and/or activity of components of the signal transduction pathway of T1R3, or the activity of the signal transduction pathway itself can be assayed.

The ability of a test molecule to modulate the activity of T1R3 may be measured using standard biochemical and physiological techniques. Responses such as activation or suppression of catalytic activity, phosphorylation or dephosphorylation of T1R3 and/or other proteins, activation or modulation of second messenger production, changes in cellular ion levels, association, dissociation or translocation of signaling molecules, or transcription or translation of specific genes may be monitored. In non-limiting embodiments of the invention, changes in intracellular Ca²⁺levels may be monitored by the fluorescence of indicator dyes such as indo, fura, etc. Additionally, changes in cAMP, cGMP, IP₃, and DAG (diacylglycerol) levels may be assayed. In yet another embodiment, activation of adenylyl cyclase-, guanylyl cyclase-, protein kinase A- and Ca²⁺-sensitive release of neurotransmitters may be measured to identify compounds that modulate T1R3 signal transduction. Further, changes in membrane potential resulting from modulation of the T1R3 channel protein can be measured using a voltage clamp or patch recording methods.

In yet another embodiment according to the disclosure, a microphysiometer can be used to monitor cellular activity. For example, after exposure to a test compound, cell lysates can be assayed for increased intracellular levels of Ca²⁺and activation of calcium-dependent downstream messengers such as adenylyl cyclase, protein kinase A or cAMP. The ability of a test compound to increase intracellular levels of Ca²⁺, activate protein kinase A or increase cAMP levels compared to those levels seen with cells treated with a vehicle control, indicates that the test compound acts as an agonist (i.e., a T1R3 activator) and induces signal transduction mediated by the T1R3 expressed by the host cell. A test compound is identified as an inhibitor based upon its ability to counteract the stimulation by sweet or umami agonist of the T1R2/3 and/or T1R1/3 receptors. An inverse agonist would be detected based upon its ability to produce an inverse response in comparison to control tastants agonists.

In a specific embodiment of the invention, levels of cAMP can be measured using constructs containing the cAMP responsive element linked to any of a variety of different reporter genes. Such reporter genes may include, but are not limited to, chloramphenicol acetyltransferase (CAT), luciferase, β-glucuronidase (GUS), growth hormone, or secreted placental alkaline phosphatase (SEAP). Such constructs are introduced into cells expressing T1R3, thereby providing a recombinant cell useful for screening assays designed to identify modulators of T1R3 activity.

Following exposure of the cells to the test compound, the level of reporter gene expression may be quantitated to determine the test compound's ability to regulate T1R3 activity. Alkaline phosphatase assays are particularly useful in the practice of the invention as the enzyme is secreted from the cell. Therefore, tissue culture supernatant may be assayed for secreted alkaline phosphatase. In addition, alkaline phosphatase activity may be measured by calorimetric, bioluminescent or chemilumenscent assays, such as those described in Bronstein, I. et al. (1994, Biotechniques 17: 172-177). Such assays provide a simple, sensitive easily automatable detection system for pharmaceutical screening.

Additionally, to determine intracellular cAMP concentrations, a scintillation proximity assay (SPA) may be utilized (SPA kit is provided by Amersham Life Sciences, Illinois). The assay utilizes ¹²⁵I-labeled cAMP, an anti-cAMP antibody, and a scintillant-incorporated microsphere coated with a secondary antibody. When brought into close proximity to the microsphere through the labeled cAMP-antibody complex, ¹²⁵I will excite the scintillant to emit light. Unlabeled cAMP extracted from cells competes with the ¹²⁵I-labeled cAMP for binding to the antibody and thereby diminishes scintillation. The assay may be performed in 96-well plates to enable high-throughput screening and 96 well-based scintillation counting instruments such as those manufactured by Wallac or Packard may be used for readout.

In yet another embodiment of the invention, levels of intracellular Ca²⁺can be monitored using Ca²⁺-indication dyes, such as Fluo-3 and Fura-Red, using methods such as those described in Komuro and Rakic, 1998, In: The Neuron in Tissue Culture. L. W. Haymes, Ed. Wiley, New York.

Test activators that activate T1R3, identified by any of the above methods, may be subjected to further testing to confirm their ability to induce a sweetness perception. Test inhibitors that inhibit the activation of T1R3 by sweet tastants, identified by any of the above methods, may then be subjected to further testing to confirm their inhibitory activity. The ability of the test compound to modulate the activity of the T1R3 receptor may be evaluated by behavioral, physiologic, or in vitro methods.

For example, assuming test animals prefer sweet tastes to non-sweet tastes, a behavioral study may be performed where a test animal may be offered the choice of consuming a composition comprising the putative T1R3 activator and the same composition without the added compound. A preference for the composition comprising a test compound, indicated, for example, by greater consumption, would have a positive correlation with activation of T1R3 activity. Additionally, lack of preference by a test animal for food containing a putative inhibitor of T1R3 in the presence of a sweetener would have a positive correlation with the identification of a sweetness inhibitor.

In addition to cell-based assays, non-cell based assay systems may be used to identify compounds that interact with (e.g., bind to) T1R3. Such compounds may act as antagonists or agonists of T1R3 activity and may be used to regulate sweet taste perception.

To this end, soluble T1R3 may be recombinantly expressed and utilized in non-cell-based assays to identify compounds that bind to T1R3. The recombinantly expressed T1R3 polypeptides or fusion proteins containing one or more of the domains of T1R3 prepared as described herein can be used in the non-cell-based screening assays. For example, peptides corresponding to the amino-terminal domain that is believed to be involved in ligand binding and dimerization, the cysteine-rich domain and/or the transmembrane spanning domains of T1R3, or fusion proteins containing one or more of the domains of T1R3 can be used in non-cell-based assay systems to identify compounds that bind to a portion of T1R3; such compounds may be useful to modulate the signal transduction pathway of T1R3. In non-cell-based assays the recombinantly expressed T1R3 may be attached to a solid substrate such as a test tube, microtiter well or a column, by means well known to those in the art (see Ausubel et al., supra). The test compounds are then assayed for their ability to bind to T1R3.

The T1R3 protein may be one which has been fully or partially isolated from other molecules, or which may be present as part of a crude or semi-purified extract. As a non-limiting example, the T1R3 protein may be present in a preparation of taste receptor cell membranes. In particular embodiments of the invention, such taste receptor cell membranes may be prepared as set forth in Ming, D. et al., 1998, Proc Natl Sci U.S.A. 95:8933-8938, incorporated by reference herein. Specifically, bovine circumvallate papillae (“taste tissue”, containing taste receptor cells), may be hand-dissected, frozen in liquid nitrogen, and stored at −80° C. prior to use. The collected tissues are then homogenized with a Polytron homogenizer (three cycles of 20 seconds each at 25,000 RPM) in a buffer containing 10 mM Tris at pH 7.5, 10% vol/vol glycerol, 1 mM EDTA, 1 mM DTT, 10 μg/μl pepstatin A, 10 μg/μl leupeptin, 10 μg/μl aprotinin, and 100 μM 4-(2-amino ethyl)benzene sulfonyl fluoride hydrochloride After particulate removal by centrifugation at 1,500×g for 10 minutes, taste membranes are collected by centrifugation at 45,000×g for 60 minutes. The pelleted membranes may then be rinsed twice, re-suspended in homogenization buffer lacking protease inhibitors, and further homogenized by 20 passages through a 25-gauge needle. Aliquots are then either flash-frozen or stored on ice until use. As another non-limiting example, the taste receptor is derived from recombinant clones (see Hoon, M. R. et al., 1999 Cell 96, 541-551).

Assays may also be designed to screen for compounds that regulate T1R3 expression at either the transcriptional or translational level. In one embodiment, DNA encoding a reporter molecule can be linked to a regulatory element of the T1R3 gene and used in appropriate intact cells, cell extracts or lysates to identify compounds that modulate T1R3 gene expression. Appropriate cells or cell extracts are prepared from any cell type that normally expresses the T1R3 gene, thereby ensuring that the cell extracts contain the transcription factors required for in vitro or in vivo transcription. The screen is used to identify compounds that modulate the expression of the reporter construct. In such screens, the level of reporter gene expression is determined in the presence of the test compound and compared to the level of expression in the absence of the test compound.

To identify compounds that regulate T1R3 translation, cells or in vitro cell lysates containing T1R3 transcripts are tested for modulation of T1R3 mRNA translation. To assay for inhibitors of T1R3 translation, test compounds are assayed for their ability to modulate the translation of T1R3 mRNA in in vitro translation extracts.

In addition, compounds that regulate T1R3 activity are identified using animal models. Behavioral, physiological, or biochemical methods are used to determine whether T1R3 activation has occurred. In some embodiments, behavioral and physiological methods are practiced in vivo. As an example of a behavioral measurement, the tendency of a test animal to voluntarily ingest a composition, in the presence or absence of test activator, is measured. If the test activator induces T1R3 activity in the animal, the animal is expected to experience a sweet taste, which would encourage it to ingest more of the composition. If the animal is given a choice of whether to consume a composition containing a sweet tastant only (which activates T1R3) or a composition containing a test inhibitor together with a sweet tastant, it would be expected to prefer to consume the composition containing sweet tastant only. Thus, the relative preference for a compound or composition demonstrated by the animal directly correlates with the activation of the T1R3 receptor by the compound or composition.

Physiological methods include nerve response studies, which may be performed using a nerve operably joined to a taste receptor cell containing tissue, in vivo or in vitro. Since exposure to sweet tastant which results in T1R3 activation is expected to result in an action potential in taste receptor cells that is then propagated through a peripheral nerve, measuring a nerve response to a sweet tastant is an indirect measurement of T1R3 activation. An example of nerve response studies performed using the glossopharyngeal nerve are described in Ninomiya, Y., et al., 1997, Am. J. Physiol (London) 272:R1002-R1006.

The assays described herein can identify compounds which modulate T1R3 activity. For example, compounds that affect T1R3 activity include, but are not limited to, compounds that bind to T1R3 or modulate T1R3 activity through binding to a co-receptor of T1R3 such as T1R2 or T1R1, and either activate signal transduction (agonists) or inhibit (e.g., block) activation (antagonists). Compounds that affect T1R3 gene activity (by affecting T1R3 gene expression, including molecules, e.g., proteins or small organic molecules, that affect transcription or interfere with splicing events so that expression of the full-length or the truncated form of the T1R3 can be modulated) can also be identified using the screens of the invention. It should be noted, however, that the assays described can also identify compounds that modulate T1R3 signal transduction (e.g., compounds that affect downstream signaling events, such as inhibitors or enhancers of G protein activities which participate in transducing the signal activated by tastants binding to their receptor). The identification and use of such compounds, which affect signaling events downstream of T1R3 and thus modulate effects of T1R3 on the perception of taste, are within the scope of the disclosure.

The compounds which may be screened in accordance with the invention include, but are not limited to, small organic or inorganic compounds, peptides, antibodies and fragments thereof, and other organic compounds (e.g., peptidomimetics) that bind to T1R3 and either mimic the activity triggered by the natural tastant ligand (i.e., agonists) or inhibit the activity triggered by the natural ligand (i.e., antagonists). Such compounds include naturally occurring compounds such as those present in fermentation broths, cheeses, plants, and fungi, for example.

Modulator compounds may include, but are not limited to, peptides such as soluble peptides, including but not limited to members of random peptide libraries (see, e.g., Lam, K. S. et al., 1991, Nature 354:82-84; Houghten, R. et al., 1991, Nature 354:84-86); and combinatorial chemistry-derived molecular libraryies made of D- and/or L-configuration amino acids, phosphopeptides (including, but not limited to, members of random or partially degenerate, directed phosphopeptide libraries; (see, e.g., Songyang, Z. et al., 1993, Cell 72:767-778), antibodies (including, but not limited to, polyclonal, monoclonal, humanized, anti-idiotypic, chimeric or single-chain antibodies, and Fab, F(ab′)₂ and Fab expression library fragments, and epitope binding fragments thereof), and small organic molecules, such as those described herein, or inorganic molecules.

Other compounds that are screened in accordance with the invention include, but are not limited to, small organic molecules, such as those described herein, that affect the expression of the T1R3 gene or some other gene (e.g., co-receptors T1R1, T1R2, calcium sensing receptor CaSR, metabotropic glutamate receptor and other receptors that can dimerize or multimerize with T1R3) involved in the T1R3 signal transduction pathway (e.g., by interacting with the regulatory region or transcription factors involved in gene expression); or such compounds that affect the activity of the T1R3 or the activity of some other intracellular factor involved in the T1R3 signal transduction pathway, such as, for example, a T1R3 associated G-protein, G-alpha gustducin, G-beta3, G-gammal3.

Compositions Containing Modulators of hT1R3 and their Uses

Compounds and/or compositions of the present disclosure, as well as methods of using the same, are provided that act as sweet taste modulators, as well as a nutraceutical and/or a pharmaceutical to the recipient.

The T1R3 modulators of the disclosure are included in pharmaceuticals or therapeutic compositions according to one embodiment of the invention. In one embodiment a T1R3 modulator of the present invention may be administered by injection, such as intravenous, intramuscular, or intraperitoneal injection. In another embodiment, a T1R3 modulator of the disclosure is administered orally. PCT/US2008/073756, which is hereby incorporated by reference in its entirety, describes formulations and methods of administration of T1R3 modulators.

Compositions comprising a T1R3 modulator of the disclosure comprise one or more pharmaceutically acceptable carriers.

Single or multiple administrations of the compositions can be carried out with the dose levels and pattern being selected by the treating physician. For the prevention or treatment of disease, the appropriate dosage will depend on the type of disease to be treated, as described above, the severity and course of the disease, whether the therapeutic is administered for preventive or treatment purposes, previous therapy, the patient's clinical history and response to the therapeutic, and the discretion of the attending physician.

The pharmaceutical composition may further comprise a pharmaceutically acceptable carrier, diluent, salt, buffer, or excipient. The pharmaceutical composition can be used for treating the above-defined disorders. The pharmaceutical composition of the invention may be a solution or a lyophilized product. Solutions of the pharmaceutical composition may be subjected to any suitable lyophilization process.

As an additional aspect, the disclosure includes kits which comprise a composition of the invention packaged in a manner that facilitates its use for administration to subjects. In one embodiment, such a kit includes a compound or composition described herein (e.g., a composition comprising a T1R3 modulator), packaged in a container such as a sealed bottle or vessel, with a label affixed to the container or included in the package that describes use of the compound or composition in practicing the method. In one embodiment, the kit contains a first container having a composition comprising a T1R3 modulator and a second container having a physiologically acceptable reconstitution solution for the composition in the first container. In one aspect, the compound or composition is packaged in a unit dosage form. The kit may further include a device suitable for administering the composition according to a specific route of administration. Preferably, the kit contains a label that describes use of the therapeutic protein or peptide composition.

The disclosure provides for methods of inducing a sweet taste resulting from contacting a taste tissue of a subject with a sweet tastant, comprising administering to the subject an effective amount of a T1R3 activator, such as a T1R3 activator identified by measuring T1R3 activation as described herein. The present invention also provides for methods of inhibiting the sweet taste of a composition, comprising incorporating, in the composition, an effective amount of a T1R3 inhibitor. An “effective amount” of the T1R3 inhibitor is an amount that subjectively decreases the perception of sweet taste and/or that is associated with a detectable decrease in T1R3 activation as measured by one of the above assays.

The disclosure further provides for a method of producing the perception of a sweet taste by a subject, comprising administering, to the subject, a composition comprising a compound that activates T1R3 activity, such as a sweetness activator identified as described herein. The composition comprises an amount of activator that is effective in producing a taste recognized as sweet by a subject.

Accordingly, the disclosure provides for compositions comprising sweetness activators and sweetness inhibitors. Such compositions include any substances which may come in contact with taste tissue of a subject, including but not limited to foods, beverages, pharmaceuticals, dental products, cosmetics, and wetable glues used for envelopes and stamps.

In one set of embodiments of the disclosure, T1R3 activators are utilized as food or beverage sweetners. In such instances, the T1R3 activators of the invention are incorporated into foods or beverages, thereby enhancing the sweet flavor of the food or beverage without increasing the carbohydrate content of the food.

In another embodiment of the disclosure, a sweetness activator is used to counteract the perception of bitterness associated with a co-present bitter tastant. In these embodiments, a composition of the invention comprises a bitter tastant and a sweetness activator, where the sweetness activator is present at a concentration which inhibits bitter taste perception. For example, the concentration of sweetness activator in the composition is adjusted until perceived bitterness is at an acceptable level.

The disclosure is used to improve the taste of foods by increasing the perception of sweetness or by decreasing or eliminating the aversive effects of bitter tastants. If a bitter tastant is a food preservative, the T1R3 activators of the invention may permit or facilitate its incorporation into foods, thereby improving food safety. For foods administered as nutritional supplements, the incorporation of T1R3 activators of the invention may encourage ingestion, thereby enhancing the effectiveness of these compositions in providing nutrition or calories to a subject.

The T1R3 activators of the invention are also incorporated into medical and/or dental compositions. Certain compositions used in diagnostic procedures have an unpleasant taste, such as contrast materials and local oral anesthetics. The T1R3 activators of the disclosure are used to improve the comfort of subjects undergoing such procedures by improving the taste of compositions. In addition, the T1R3 activators of the invention may be incorporated into pharmaceutical compositions, including tablets and liquids, to improve their flavor and to improve patient compliance (particularly where the patient is a child or a non-human animal).

The T1R3 activators of the disclosure are included in cosmetics to improve their taste features. For example, but not by way of limitation, the T1R3 activators of the disclosure are incorporated into face creams and lipsticks. In addition, the T1R3 activators of the disclosure are incorporated into compositions that are not traditional foods, beverages, pharmaceuticals, or cosmetics, but which contact taste membranes. Examples include, but are not limited to, soaps, shampoos, toothpaste, denture adhesive, glue on the surfaces of stamps and envelopes, and toxic compositions used in pest control (e.g., rat or cockroach poison).

It will be appreciated that the methods of the present disclosure may be useful in fields of human medicine and veterinary medicine. Thus, the “subject” to be treated may be a mammal, preferably human or other animal. For veterinary purposes, subjects include, for example, farm animals such as cows, sheep, pigs, horses and goats, companion animals such as dogs and cats, exotic and/or zoo animals, laboratory animals including mice, rats, rabbits, guinea pigs, and hamsters; and poultry such as chickens, turkey, ducks and geese.

The following examples are provided for illustration and are not intended to limit the scope of the disclosed subject matter.

### Example 1

### Phenoxy Herbicides and Fibrates Potently Inhibit the Human T1R3 Chemosensor Receptor

Materials and Methods

Chemicals:

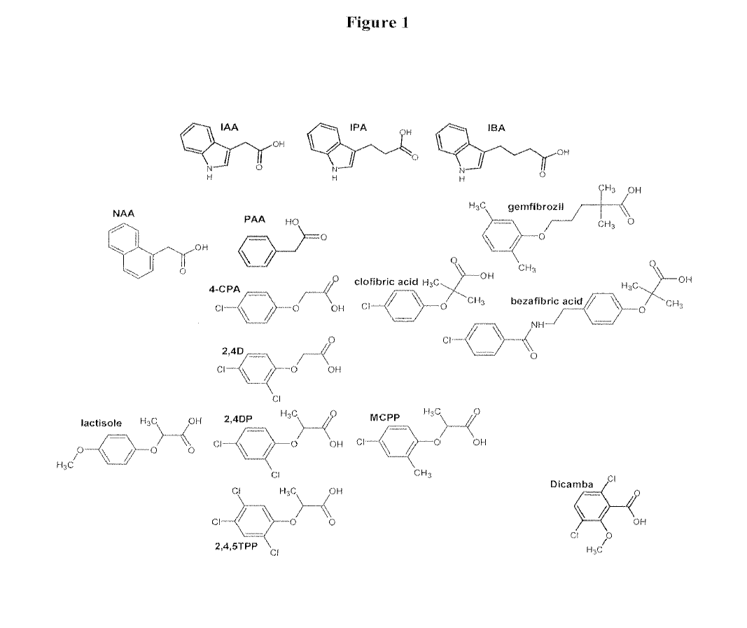

Chemicals were obtained from Sigma-Aldrich. Purity of all chemicals used was at least >98%. Fibrates and herbicides were used as acids, their active forms, as shown in FIG. 1. 10 mM solutions were prepared in DMSO or water according to the compounds' solubility. Chemical structures were generated using the ChemSketch2.0 software.

DNA Constructs:

Human and mouse T1R2 and T1R3 chimeras, and the G 16-gust44 were prepared in pcDNA3 vector as described in (Xu, H., et al., Proc Natl Acad Sci USA, 2004, 101, 14258-14263; and Jiang, P. et al., J Biol Chem 2005, 280, 15238-15246; each hereby incorporated by reference in their entirety). The integrity of all DNA constructs was confirmed by sequencing.

Heterologous Calcium Assay for Sweet-Sensing Receptors:

HEK293E cells were cultured at 37° C. in Optimem GlutaMAX culture medium (Invitrogen) supplemented with 4% dialyzed fetal bovine serum. Cells were transfected with the DNA constructs using lipofectamine-2000 according to the manufacturer's protocol (Invitrogen). Briefly, cells were seeded onto 96-well poly-D-lysine plates (Corning) at about 12,500 cells/well 18 hours prior to transfection; and co-transfected with plasmid DNAs encoding T1R5 and G 16-gust44 (0.1 μg total DNA/well; 0.2 μl lipofectamine/well). After 24 hours, the transfected cells were washed once with the culture medium and incubated for another 24 hours. The cells were washed with HBSS supplemented with 20 mM Hepes (HBSS-H), loaded with 3 μM Fluo-4AM (Molecular Probe) in HBSS-H buffer, incubated for 1.5 hours at room temperature, and then washed with HBSS-H and maintained in HBSS-H at 25° C. The plates of dye-loaded transfected cells were placed into a FlexStation II apparatus (Molecular Devices) to monitor fluorescence (excitation, 488 nm; emission, 525 nm; cutoff, 515 nm). Sweeteners were added 30 seconds after the start of the scan at 2× concentration in 50 μl of HBSS-H while monitoring fluorescence for an additional 200 seconds at 2 seconds intervals. Where tested, inhibitors were added along with sweeteners to the cells.

Data Analysis of Calcium Responses:

After obtaining a calcium mobilization trace for each sample, calcium responses to sweeteners were quantified as the percentage of change (peak fluorescence-baseline fluorescence level, denoted as F) from baseline fluorescence level (denoted as F); ΔF/F. Peak fluorescence intensity occurred about 20-30 seconds after the addition of agonists. Typically, wild-type sweet receptors (T1R2+T1R3) along with G 16-gust44 show calcium signal increases (ΔF/F) from 40 to 100% of the basal signal F. Buffer alone evokes no significant response from transfected cells and sweeteners plus inhibitors evoke no significant responses from parent cells (3%≦F/F≦3%, S.E). The data were expressed as the mean±S.E. of quadruplicate or sextuplicate of the F/F values, or were normalized as described. The bar graph and curving-fitting routines were carried out using Graph-Pad Prism 3.0 (GraphPad Software, Inc.).

Inhibition of T1R3

FIG. 1 provides the chemical structures of lactisole, as well as several phenoxy-herbicides and fibrates.

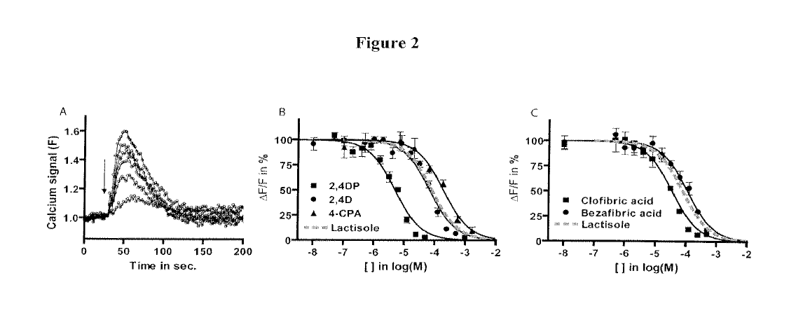

To determine if phenoxy-herbicides and fibrates had inhibitory properties, a cell-based assay with HEK 293 cells expressing the human sweet receptor composed of hT1R3 and hT1R2 was used. Activation of the receptor by sugars or sweeteners was detected by intracellular calcium mobilization (FIG. 2A). Heterologously expressed human sweet receptors were inhibited by herbicides with chlorinated and/or methylated phenoxy-propionic acid motifs (2,4DP, MCPP and 2,4,5TPP) (IC50s from 5-12 μM) even more potently than by lactisole (IC50 70 μm) (FIG. 2BC, Table 1). Bi-chlorinated phenoxy-acetic acid 2,4D also strongly inhibited the human receptor (IC50 70 μM), while the mono-chlorinated 4-CPA and non-phenoxy-herbicides PAA and NAA inhibited only at higher concentrations (IC50s from 142 to >500 μM) (FIG. 2AB). Indole-type auxins (IPA, IAA, IBA) at up to 500 μM did not inhibit the human sweet receptor (Table 1). Dicamba (3,6 dichloromethoxybenzoic acid), and trichloro (2, ¾, ⅚) benzoic acids at up to 1 mM did not inhibit T1R3.

Table 1:

IC₅₀ inhibition values of several phenoxyauxins and fibrates on the sweet-sensing receptor. Abbreviations: IPA: 3-(1H-indol-3-yl)propanoic acid. IAA: 1H-indol-3-ylacetic acid. IBA: 4-(1H-indol-3-yl)butanoic acid. PAA: phenylacetic acid. NAA: naphthalen-1-ylacetic acid. 4-CPA: (4-chlorophenoxy)acetic acid. 2,4,5TPP: 2-(2,4,5-trichlorophenoxy) propanoic acid. 2,4D: (2,4-dichlorophenoxy)acetic acid. 2,4DP: 2-(2,4-dichlorophenoxy) propanoic acid. MCPP: 2-(4-chloro-2-methylphenoxy) propanoic acid. Lactisole: 2-(4-methoxyphenoxy) propanoic acid. Clofibric acid: 2-(4-chlorophenoxy)-2-methylpropanoic acid. Gemfibrozil: 5-(2,5-dimethylphenoxy)-2,2-dimethylpentanoic acid. Bezafibric acid: 2-[4-(2-{[(4-chlorophenyl)carbonyl]amino}ethyl)phenoxy]-2-methylpropanoic acid.

| COMPOUND | IC50 (μM (SD) |
|---|---|
| Lactisole | 70 (13) |
| IPA | >1000 |
| IAA | >1000 |
| IBA | >1000 |
| PAA | >500 |
| NAA | 233 (82) |
| 4-CPA | 142 (19) |
| 2,4D | 50 (10) |
| 2,4DP | 5.3 (1.3) |
| 2,4,5TPP | 5.7 (1.7) |
| MCPP | 12 (3) |
| Clofibric acid | 28 (8) |
| Gemfibrozil | 69 (20) |
| Bezafilbric acid | 100 (11) |

IC50 values of the tested compounds were determined from the calcium mobilization assays with heterologously expressed human T1R2+T1R3 receptor activated by 2.5 mM sucralose in the presence of increasing concentrations of inhibitors. IC50s are means±SD from at least 3 independent experiments.

Clofibric acid, bezafibric acid and gemfibrozil potently inhibited the human sweet receptor (IC50s from 30 to 100 μM) (FIG. 2AB, Table 1).

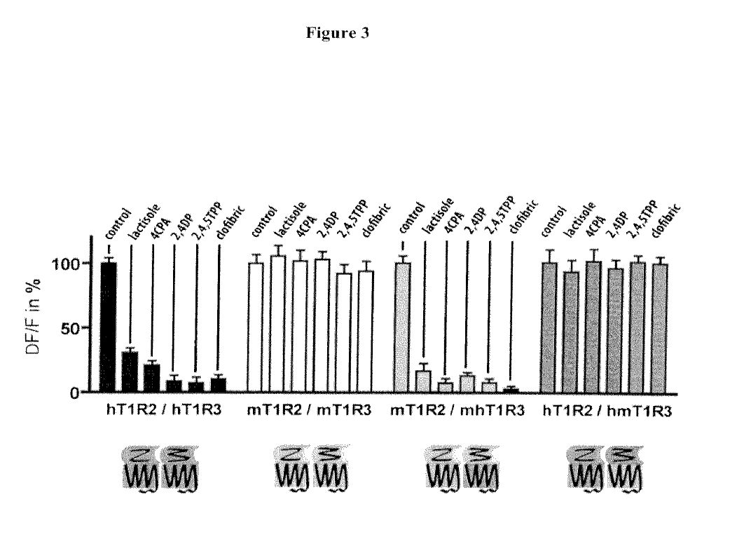

The (human) specificity of the inhibitory effect of the tested compounds was also determined. Lactisole inhibits the human sweet taste receptor; however, it has no effect on the mouse receptor. The phenoxy-herbicides and fibrates displayed the same species-specificity in inhibiting the human but not the mouse sweet receptor (FIG. 3). Assays with human/mouse chimeric sweet receptors showed that the ability of phenoxy-auxins and fibrates to inhibit the sweet receptor depended on the presence of the human form of the seven-transmembrane domain of T1R3 (FIG. 3).

Discussion

As shown herein, widely used herbicides 2,4DP, 2,4D and MCPP potently inhibit the human T1R2+T1R3 receptor. Clofibrate and related anti-lipid drugs such as bezafibrate and gemfibrosil also strongly inhibit the human T1R2+T1R3 receptor. These compounds act specifically on the human and not the rodent form of the sweet receptor. Furthermore, it is the transmembrane portion of human T1R3 that is targeted by these compounds. Because only old world monkeys and primates (including humans) have similar T1R3 receptors that would respond to lactisole and related compounds (Jiang, supra; Nofre, C., et al., Chem Senses 1996, 21, 747-762; Wang, Y., et al., BMC Physiol 2009, 9, 1) most animal models would not have shown any effects through their T1R3 receptors, either in taste cells or gut endocrine cells.

From the measured IC50s of fibrates and phenoxy herbicides on T1R3, longer and more branched aliphatic chains in fibrates weaken the inhibitory activity toward the T1R3. Varying the length or branching of the chain is expected to affect the level of inhibition of T1R3: for example 2, 4DP is 10-fold more potent than is 2,4D. The only difference between the two structures is that propionic acid is replaced by acetic acid (FIG. 1). The inhibitory activity toward T1R3 is also affected by the modifications of the aromatic portion of the compounds. Naphthalene acetic acid, NAA, shows modest activity while its phenyl counterpart, PAA, is very weak, and the structures with indole rings (IAA, IPA) have no activity. Di-chloro substitutions (ortho or para) on the phenoxy group appear to improve T1R3 inhibitory potency in comparison to one single para-methoxy, single para-chloro substitutions, or double ortho-methyl/para-chloro substitutions (e.g. 2,4DP is 10× more potent than lactisole; and 2,4DP, and 2,4D gains 2-3-fold potency over MCPP and 4,CPA respectively). Tri-chloro substitutions do not have any further effect on T1R3 inhibitory activity (e.g. 2,4DP and 2,4,5TPP have about the same potency). The phenoxy-motif seems obligatory as its absence could be the reason that dicamba (and trichlorinated benzoic acid) do not inhibit T1R3, whereas 2,4D and 2,4,5TPP do.

T1R3's role as a critical component of sweet and umami receptors in taste cells is well established (Zhao, supra; and Max, supra). T1R3, like gustducin, has also been implicated in glucose-sensing functions of gut endocrine cells and thereby in glucose homeostasis (Jang, supra). From psychophysical self-experimentation conducted by one of the inventors, it was found that clofibric acid potently inhibited both sweet and umami (MSG) taste in vivo.

With respect to fibrates, the results described herein show that IC50s (30 to 100 μM) of clofibric and bezafibric acids for inhibiting the sweet receptor are comparable to their EC50s (50-55 μM) for activating PPAR-alpha (Willson, supra). Based on this information, the plasma level of clofibric acid attained in the course of the fibrate treatment would be sufficient to systemically block the T1R3 receptor. Thus, T1R3-containing receptors are expected to be an important biological target of fibrates and could mediate certain of their effects on lipid metabolism and glucose homeostasis.

T1R3 receptors are now known to be expressed in a number of tissues: taste cells, endocrine cells in the gastrointestinal tract, and cells in pancreas and testes (Toyono, supra; Taniguchi, K., J Vet Med Sci 2004, 66, 1311-1314; Kiuchi, S., et al., Cytogenet Genome Res 2006, 115, 51-61; and Nakagawa, Y., et al., PLoS ONE 2009, 4, e5106). In rodents, T1R3, gustducin and other signaling molecules previously found in taste cells have been shown to be present in gut endocrine cells and implicated in nutrient sensing and regulation of glucose metabolism through release of intestinal hormones (Jang, supra, Egan, supra, and Margolskee, supra). To date, comparable studies in humans or old-world monkeys have not been conducted. The results presented herein exemplify the need for testing chemicals intended for human use and/or consumption on tissues, cells and organisms with pharmacological targets similar or identical to humans. Based on the results described herein, compounds that selectively act on T1R3 but not PPAR can be identified and prove useful in the treatment of metabolic syndrome, obesity and type II diabetes and other carbohydrate and lipid metabolic disorders or diseases.

### Example 2

### Animal Model for Screening Inhibitory Compounds Binding to Transmembrane Domain

Preparation of Constructs

Two chimeric T1R3 receptors were constructed. One (designated ½) contains the entire extracellular domain of the mouse T1R3, while the intracellular end-portion (described also as transmembrane domain) of the mouse receptor (starting at amino acid 567) was replaced by human sequence (starting at amino acid 568). The second construct (designated 1/10) has only trans-membrane domain 5 and 6 of the mouse T1R3 replaced by the human sequence which is the part required for lactisole, fibrates and phenoxyauxin-herbicide's binding. Both receptors express well in cell-based assays and pair with mouse T1R2 receptor, forming a fully-functional sweet receptor that is inhibited by lactisole. Studies with analogous T1R3 chimeric receptors have been published (Xu 2004, Jiang 2005). The entire human T1R3 receptor cannot be used because it does not form, for unknown reasons, a functional receptor with mouse T1R2. However, the exact part that needs to be replaced in order to create the humanized binding pocket can be rather short, i.e. domain 5+6 out of 7 domains or even several amino acids of human T1R3.

In addition, two DNA constructs were prepared containing the chimeric receptors together with an IRES (internal ribosomal entry site) and eGFP (enhanced green fluorescent protein). In these constructs, the receptors and GFP are expressed from an ubiquitous promoter which allowed expression confirmation by GFP fluorescence and evaluation of the receptor's functionality in cell based assays before proceeding with cloning (under 13 kb T1R3 promoter). The 13 kb promoter was previously successfully used to drive GFP in transgenic animals (Damak S, et al., BMC Neurosci., 2008; 9:96).

Finally, the chimeric receptors-IRES-GFP cassettes were cloned under the 13 kb long T1R3 promoter.

In Vivo Studies

The ½ construct was injected into fertilized eggs of T1R3 knock-out (KO) and wild-type (WT) mice. 6 DNA positive founders on the T1R3 null background, and 9 with WT background have been obtained.

The founders on the T1R3 null background are evaluated to determine if they gained the ability to detect sugars and/or sweeteners (parental T1R3KO animals cannot detect sugars and sweeteners). A non-invasive determination is performed to identify which founders are expressing the transgene by examining their sweet and umami preference. The inhibition of the sweet preference by lactisole is also tested in this way. By further breeding the selected animals with T1R3 null mice, lines are established where the m/hT1R3 is the only functional T1R3 receptor.

Behavioral testing the transgenics (relative to WT background) is also performed to determine their preference to the artificial sweetener cyclamate. Cyclamate is not sweet to mice, because it specifically binds to the transmembrane domain of the human T1R3 receptor.

Discussion

Based on the above results and disclosure, assays to determine the blocking effect of various compounds on human T1R3 in behavioral tests are provided. Likewise, ex vivo and in vivo assays to determine changes in metabolic and hormonal parameters that are affected by stimulation or inhibition of the hT1R3 in short-term and long-term studies are also contemplated. By way of example, the release of GLP-1 and other intestinal hormones can be measured, or insulin and glucagon from pancreatic islets in short term assays. In long-term assays, the effect of human-specific inhibitors on carbohydrate and lipid metabolism, and/or effects on diet induced obesity are evaluated.

### Example 3

### Tissue Expression Using Transgenic Mice

The constructs described in Example 2 were used to prepare transgenic mice expressing the chimeric receptor (the ½ construct). GFP expression revealed that the transgene (GFP) is expressed in taste cells, and also in pancreatic islets, gut mucosal cells (enteroendocrine), and importantly, very strongly in tubular cells in testis. Expression was also observed in several areas of the brain and in retina.

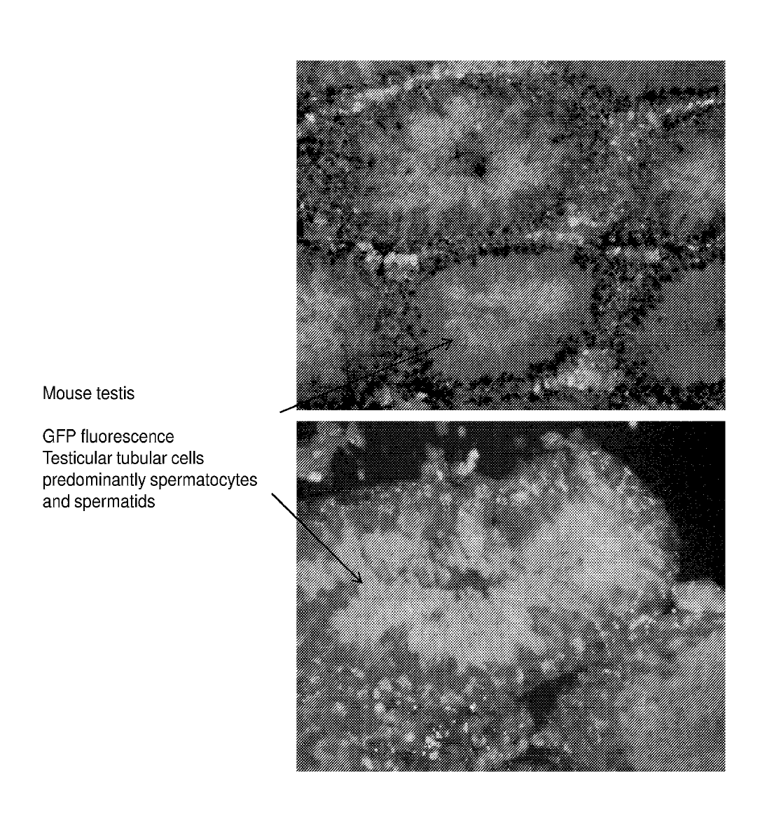

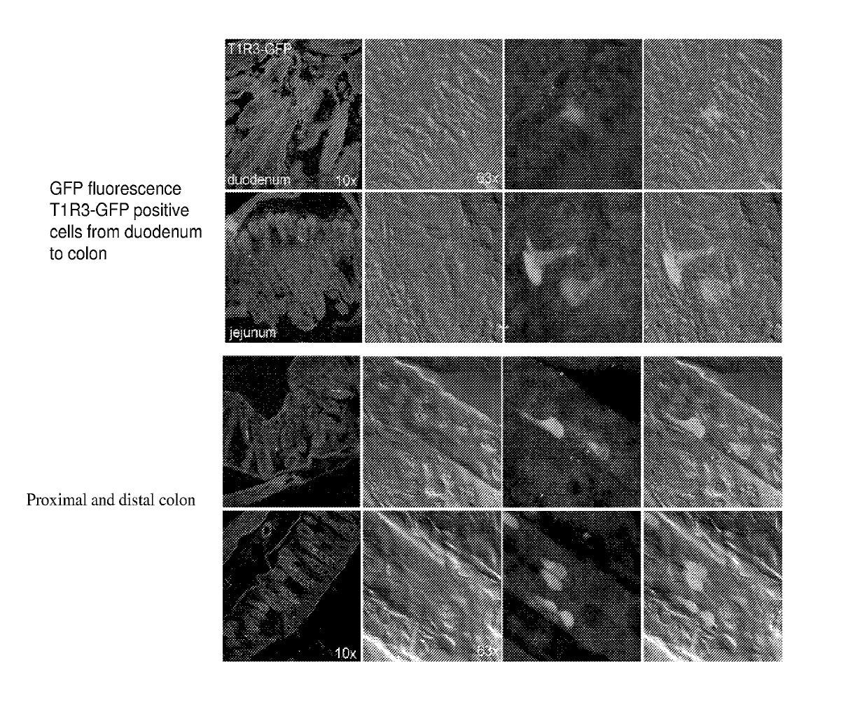

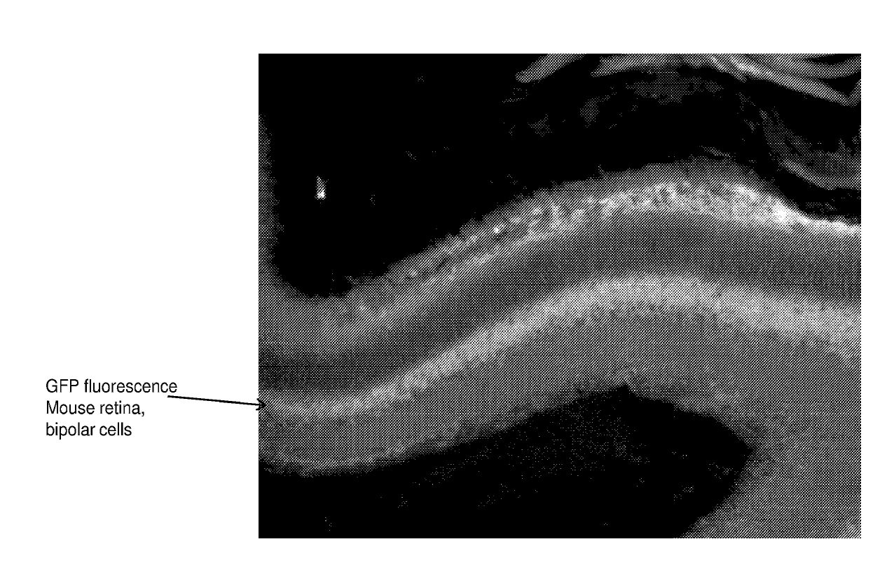

Expression of T1R3-GFP in testis of transgenic mice is shown in FIG. 8 using frozen tissue sections of testis from T1R3-mhT1R3-IRES-GFP mouse. GFP fluorescence is strong in spermatocytes and spermatids inside the testicular tubules (arrows). The signal on the periphery of tubules is a nonspecific autofluorescence visible in any WT mouse tissue. Expression of T1R3-GFP in gut of transgenic mice is shown in FIG. 9 using frozen tissue sections of duodenum, jejunum, proximal and distal colon (clockwise) from T1R3-mhT1R3-IRES-GFP mouse. Solitary mucosal cells are visible in all parts of the gut. Expression of T1R3-GFP in retina of transgenic mice is shown in FIG. 10 using frozen tissue sections of retina from T1R3-mhT1R3-IRES-GFP mouse. GFP fluorescence is present in the layer of bipolar cells located between the retinal receptor cells and ganglion cells.

### Example 4

### A Mouse Model Conditionally Expressing a Humanized Form of the T1R3 Receptor

Examples 1, 2,3 can be extended to include generation of a mouse model where humanized T1R3 receptor can be expressed specifically in selected tissues. Using this new model, inhibition the T1R3 receptor specifically in a particular organ or tissue is possible.

In this model, the DNA construct is further refined so that its activity can be switched on only in selected tissues. For example, T1R3 is normally expressed in several tissues: taste, gut, pancreas, testis, brain. Expression in only testis, or in gut, for example, is controllable in this model. In those selected organs, only the cells that normally express T1R3 will express the humanized T1R3. In all other tissues of the animal the gene will be silent. The specific activation is achieved by interbreeding of the hT1R3 knock-in line with a mouse line expressing Cre recombinase from a tissue-specific promoter. In this model, expression of the hT1R3 in the construct is blocked by presence of Lox-STOP-Lox sequence. Cre recombinase recombines the two Lox sites and thereby removes the STOP sequence, which allows the gene to be active. Only the cells that express the Cre will activate the gene. For example, by crossing the hT1R3 knock-in mouse with a mouse expressing Cre only in testis the hT1R3 will be activated exclusively in testis. The Cre itself has no biological function in mammalian cells and the Lox sequence does not naturally exist in mammalian genome.

METHODS. The DNA construct will contain the coding region of the mouse/human chimeric receptor, preceded by the LoxP-Stop-LoxP sequence (5) and neomycine resistance gene (neo) flanked with Flippase Recognition Target (FRT) sequences. Thymidine kinase (Tk) gene will be included at the 3′ end of the construct for negative selection of non-recombinant clones (see representation, below). The LoxP-Stop-LoxP sequence is designed to effectively prevent expression by multiple means, and has been used in a number of studies (Lakso M, et al., (1992) Proc Natl Acad Sci USA 15; 89(14):6232-6; and Bartholin L, et al., (2008) Genesis 46(12):724-31.) where it blocked expression of an oncogene. The LoxP-Stop-FRT-neo-FRT-LoxP-hT1R3 construct will insert between the 5′ untranslated region of the first exon of mouse T1R3 and the 3′ end and replace the entire coding region, which is only 4 kb long with very short introns. After Cre recombination the hT1R3 sequence will function as a minigene. The single LoxP sequence will remain in the 5′ untranslated region.

Standard DNA manipulation techniques will be used to produce the DNA construct. We will electroporate the linearized DNA construct into C57BL embryonic stem cells and select them with G418 (for the presence of neo)+gancyclovir (to eliminate cells with Tk). Several hundred colonies will be isolated and their DNA screened for the homologous recombination event by PCR. Identified recombinants will be further manipulated by transient expression of FLP recombinase (Flippase) from circular FLP-Pac (puromycin) vector to remove the neo which would influence expression of the hT1R3. Flippase is another recombinase able to specifically recombine the FRT sites. Selection with puromycin is very rapid and in 2 days virtually eliminates cells that do not express the puromycin resistance. The resulting colonies will be screened for the absence of neo, but presence of Lox-Stop-Lox. Creation of targeted embryonic stem (ES) cells is alo possible. Chimeric animals will be bred first with wt animals and after germline transmission the heterozygotes will then be crossed with a general “deletor line” such as EIIa-Cre (Lakso M, et al., (1996) Proc Natl Acad Sci USA 93(12):5860-5.), expressing Cre from adenovirus promoter early on in development in virtually all cells to verify the functionality of the construct.

For tissue-specific activation, the new line will be bred with animals that express Cre in a tissue of interest.

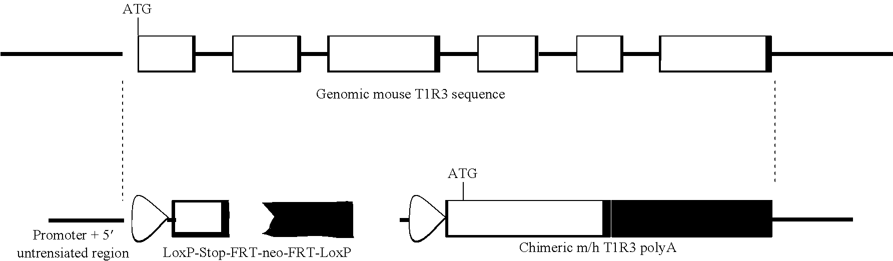

Structure of the T1R3 targeting vector. T1R3 gene structure and the targeting vector with homologous 5′ and 3′ regions are shown. After recombination in embryonic stem (ES) cells, the targeted allele will be identical to the vector. Flippase will be used to remove the neo gene flanked by the Flippase Recognition Target (FRT) sequences in ES cells. After Cre-mediated excision in animals, the T1R3 minigene will remain in the locus with one LoxP site in the 5′ untranslated region of the formerly first exon. LoxP sites are shown as triangles.

All of the compositions and/or methods disclosed and claimed herein can be made and executed without undue experimentation in light of the present disclosure. While the compositions and methods of this disclosure have been described in terms of specific embodiments, it will be apparent to those of skill in the art that variations of the compositions and/or methods and in the steps or in the sequence of steps of the method described herein can be made without departing from the concept, spirit and scope of the disclosed subject matter. More specifically, it will be apparent that certain agents which are both chemically and physiologically related may be substituted for the agents described herein while the same or similar results are achieved. All such similar substitutes and modifications apparent to those skilled in the art are deemed to be within the spirit, scope and concept of the disclosed subject matter as defined by the appended claims.

The references cited herein, to the extent that they provide exemplary procedural or other details supplementary to those set forth herein, are all specifically incorporated herein by reference.

1. A method of modulating the activity of human type I taste receptor subunit 3 (hT1R3) comprising administering to a subject an effective amount of a modulator having a structure of formula (I): 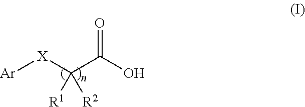
wherein Ar is an aryl group; R¹ and R² are each independently hydrogen or C₁₋₆alkyl; X is null or O; and n is 1, 2, 3, or 4, or a salt or ester thereof;
with the proviso that when R¹ is methyl, R² is hydrogen, and X is O, Ar is not 4-methoxyphenyl;
wherein said modulator binds to and modulates the activity of hT1R3.

2. The method of claim 1, wherein Ar is selected from the group consisting of phenyl, naphthyl, and imidazoyl.

3. The method of claim 2, wherein the phenyl, naphthyl, or imidazoyl is substituted with C₁₋₆alkyl, halo, or both.

4. The method of claim 3, wherein the halo is chloro.

5. The method of claim 1, wherein R¹ and R² are each hydrogen or methyl.

6. The method of claim 5, wherein R¹ and R² are both methyl.

7. The method of claim 1, wherein X is O.

8. The method of claim 1, wherein the modulator further activates peroxisome proliferators-activated receptor a.

9. (canceled)

10. The method of claim 1 wherein the modulator is selected from the group consisting of:

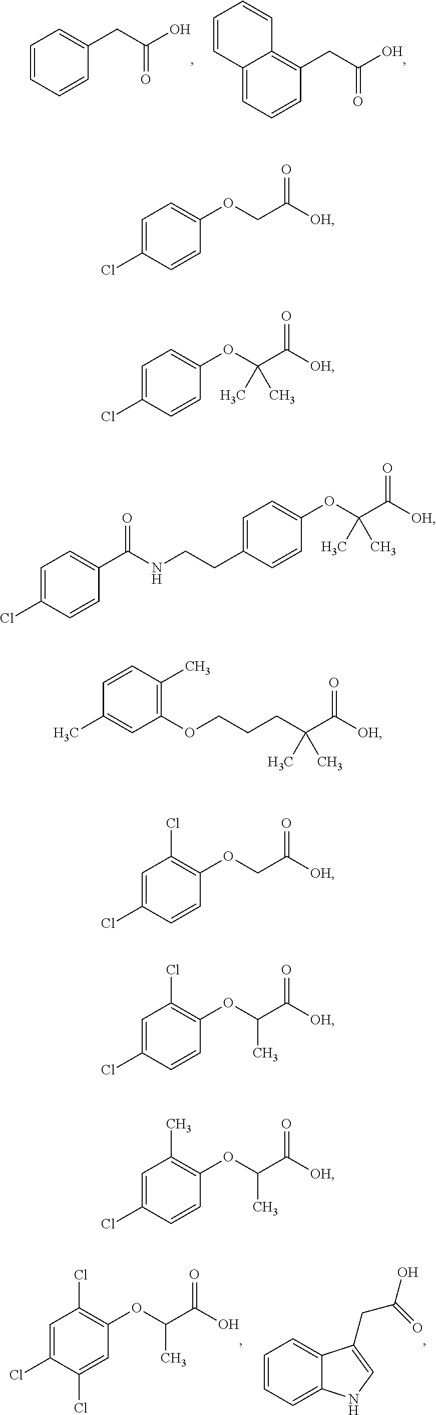

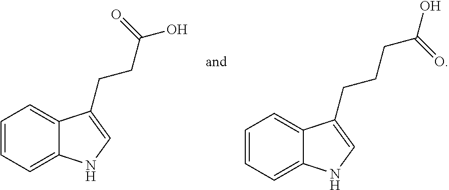

11. A method of treating a disorder associated with lipid metabolism selected from the group consisting of: hyperlipidemia, atherosclerosis, acute pancreatitis, hypercholesterolemia, said method comprising administering to a subject an effective amount of a modulator having a structure of formula (I): 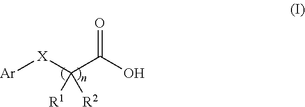
wherein Ar is an aryl group; R¹ and R² are each independently hydrogen or C₁₋₆alkyl; X is null or O; and n is 1, 2, 3, or 4, or a salt or ester thereof;
with the proviso that when R¹ is methyl, R² is hydrogen, and X is O, Ar is not 4-methoxyphenyl;
wherein said modulator binds to and modulates the activity of hT1R3, thereby treating a disorder associated with lipid metabolism.

12-20. (canceled)

21. A method of treating a disorder associated with carbohydrate metabolism selected from the group consisting of: obesity, metabolic syndrome, hyperglycemia, hypertriglyceridemia, diabetes type I, diabetes type II, and hypertension, said method comprising administering to a subject an effective amount of a modulator having a structure of formula (I): 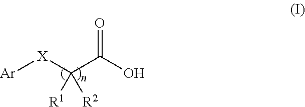
wherein Ar is an aryl group; R¹ and R² are each independently hydrogen or C₁₋₆alkyl; X is null or O; and n is 1, 2, 3, or 4, or a salt or ester thereof;
with the proviso that when R¹ is methyl, R² is hydrogen, and X is O, Ar is not 4-methoxyphenyl;
wherein said modulator binds to and modulates the activity of hT1R3, thereby treating a disorder associated with carbohydrate metabolism.

22-30. (canceled)

31. A method of treating a disorder associated with impaired carbohydrate absorption selected from the group consisting of: anorexia, bulimia, intestinal malabsorption syndromes, and celiac disease, said method comprising administering to a subject an effective amount of a modulator having a structure of formula (I): 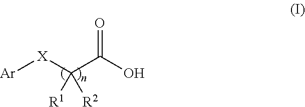
wherein Ar is an aryl group; R¹ and R² are each independently hydrogen or C₁₋₆alkyl; X is null or O; and n is 1, 2, 3, or 4, or a salt or ester thereof;
with the proviso that when R¹ is methyl, R² is hydrogen, and X is O, Ar is not 4-methoxyphenyl;
wherein said modulator binds to and modulates the activity of hT1R3, thereby treating a disorder associated with carbohydrate metabolism.

32-43. (canceled)

44. A method of screening for a modulator of hT1R3 activity comprising the steps of:
(a) contacting a cell expressing hT1R3 with a candidate compound having a structure of formula (I): 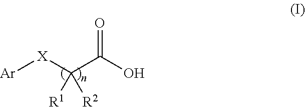
wherein Ar is an aryl group; R¹ and R² are each independently hydrogen or C₁₋₆alkyl; X is null or O; and n is 1, 2, 3, or 4, or a salt or ester thereof;
with the proviso that when R¹ is methyl, R² is hydrogen, and X is O, Ar is not 4-methoxyphenyl;
(b) measuring the activity of hT1R3;
(c) comparing the activity of hT1R3 measured in step (b) to the activity of a hT1R3 in the absence of the candidate compound; and
(d) identifying said candidate compound as a modulator of hT1R3 activity if an increase or decrease in hT1R3 activity is measured relative to the absence of said candidate compound.

45-58. (canceled)

59. A modulator of T1R3 having a structure of formula (I): 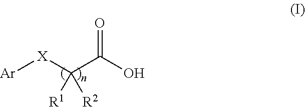
wherein Ar is an aryl group; R¹ and R² are each independently hydrogen or C₁₋₆alkyl; X is null or O; and n is 1, 2, 3, or 4, or a salt or ester thereof;
with the proviso that the modulator is not a compound selected from the group consisting of: 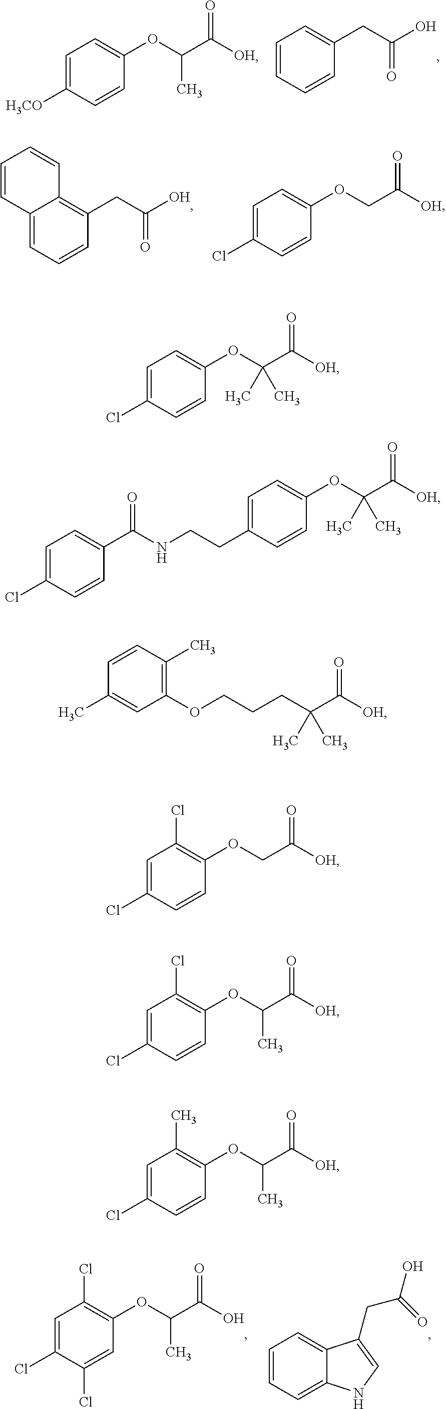 [chemical structure]
